(19) United States
(12) Patent Application Publication
(10) Pub. No.: US 2012/0077874 A1
(43) Pub. Date: Mar. 29, 2012

(54) DEVELOPMENT OF A PHYTOESTROGEN PRODUCT FOR THE PREVENTION OR TREATMENT OF OSTEOPOROSIS USING RED CLOVER

(76) Inventors: Yun Kau TAM, Edmonton (CA); Yi-Chan James LIN, Edmonton (CA); Brian Duff SLOLEY, Edmonton (CA); Chih-Yuan TSENG, Edmonton (CA)

(21) Appl. No.: 13/251,267

(22) Filed: Oct. 2, 2011

Related U.S. Application Data
(63) Continuation-in-part of application No. 13/028,136, filed on Feb. 15, 2011, now abandoned.
(60) Provisional application No. 61/304,589, filed on Feb. 15, 2010.

(51) Int. Cl. A61K 31/353; G06G 7/60; G06F 17/10; A61P 19/10

(52) U.S. Cl. 514/456; 703/11; 703/2

(57) ABSTRACT

A phytoestrogen blend was developed using a pharmaceutical platform technology to identify the time course of active components and effect time course of these components in the biophase after administration of a red clover extract. This phytoestrogen blend consists of biochanin A, daidzein, equol and genistein. The recommended daily dosage ranges from 5 to 200 mg of total isoflavone.

This application is a continuation-in-part of U.S. application Ser. No. 13/028,136, filed Feb. 15, 2011, which claims benefit of U.S. App'l No. 61/304,589, filed Feb. 15, 2010, the contents of which are incorporated herein by reference in its entirety.

### BACKGROUND OF THE INVENTION

Deficiency of estrogens during menopause can lead to a number of complications including hot flushes, reduced bone density, mood swings, etc. These symptoms are commonly treated with synthetic hormones. Although the rate of bone density reduction can be alleviated, hormone replacement therapy (HRT) (Allred, Allred et al.) was discovered to be associated with increased cardiovascular disorders in one of the largest studies of its kind (Women's health Initiative, WHI) (2004). HRT was also linked to increased risk of breast and ovarian cancer (Fernandez, Gallus et al. 2003; Gambacciani, Monteleone et al. 2003). After the WHI trial results were published, the use of HRT was reduced dramatically. Many postmenopausal women have resorted to alternative therapy because phytoestrogens are generally considered to be safe and efficacious. The use of soy and Red clover (Trifolium pratense), which are rich in phytoestrogens, has been on the rise (Beck, Rohr et al. 2005). Despite the trend, clinical trial results on phytoestrogens, however, have been equivocal (Beck, Rohr et al. 2005; Booth, Piersen et al. 2006; Wuttke, Jarry et al. 2007; Ma, Qin et al. 2008). Alternative therapy has not replaced HRT effectively. A recent study showed that the trend of women moving away from HRT has led to an alarming increase in bone fractures and it is estimated that fractures related to menopause is expected to exceed 40,000 per year in women aged 65-69 years (Gambacciani, Ciaponi et al. 2007). Since the side effects of HRT were publicized after the WHI trial, it has since been reevaluated. There is no consensus with regard to HRT's safety among the medical research community. Therefore, a much closer look at the ‘less than expected’ effects of phytoestrogens should be undertaken because the toxicity profile of this type of products is so much more favorable. In this invention, a rational design of a phytoestrogen product, which possesses appropriate clinical attributes, is revealed. Isoflavone contents in soy and red clover are different. However, none of these sources provide the optimal combination of bioactives.

The major bioactive isoflavones in soy are genistein, daidzein, glycitein and prunetin (Setchell and Cassidy 1999). They are also present in their glycoside forms. There are three classes of bioactives in red clover: isoflavones, coumestrols and lignans (Thompson, Boucher et al. 2007). The quantity of coumestrols and lignans is small; therefore, their contribution to the overall activity is likely minimal. The major isoflavones in red clover are biochanin A and formononetin (Liu, Burdette et al. 2001; Overk, Yao et al. 2005; Booth, Overk et al. 2006). Genistein and daidzein are present in minute quantities. Biochanin A and formononetin are precursors of their respective active moieties, genistein and daidzein. The conversion takes place in the intestine by intestinal flora and liver. Daidzein is converted by bacteria in the colon to form a more estrogenic metabolite, equol. In Red clover, a significant quantity of Biochanin A and formononetin is in the form of glycosides. The glycosides in soy and red clover are converted to their respective aglycones by the intestinal flora before absorption (Setchell and Cassidy 1999).

A number of clinical trials have been performed using soy or red clover and their clinical outcomes have been reviewed by a number of authors (Beck, Rohr et al. 2005; Booth, Piersen et al. 2006; Ma, Qin et al. 2008). Unfortunately, trial results are not easily comparable because most of the studies employed total isoflavones as a means of dosing. The ratios of formononetin, biochanin A, genistein, daidzein and glycitein are highly variable among products (Abrams, Griffin et al.). Since products are not standardized to individual isoflavones, there is little surprise that clinical outcomes are highly variable because potency and pharmacokinetic characteristics of each component are not the same (Table 1).

TABLE 1
Composition of Phytoestrogens in Different Commercial Products

| | Daily dosage | | | | |
|---|---|---|---|---|---|
| | (Total | Biochanin A | Formononetin | Genistein | Daidzein |
| Name | isoflavones, mg) | % wt | % wt | % wt | % wt |
| Promensil ® | 40 | 4.13 | 2.6 | 0.17 | 0.1 |
| Rimostil | 57 | 2.28 | 20.65 | 0.03 | 0.11 |
| Trinovin | 40 | 4.35 | 2.5 | 0.16 | 0.1 |
| Rotklee Activ | N.A. | 2.37 | 4.8 | 0.17 | 0.36 |
| tablets | | | | | |
| Red clover | N.A. | 2.39 | 4.64 | 0.36 | 0.82 |
| tablets | | | | | |
| Red clover | 40 | 4.6 | 2.5 | 0.13 | 0.13 |
| Isoflavones | 40 | 4.62 | 2.67 | 0.16 | 0.15 |
| Boots | | | | | |
| Menoflavon | 40-80 | 1.97 | 5.46 | 0.11 | 0.43 |

PROMENSIL® and RIMOSTIL, both manufactured by Novogen, Inc., are two highly standardized red clover extracts on the market (Table 1). They mainly contain formononetin and biochanin A, the precursors of the active moieties daidzein and genistein, respectively. PROMENSIL® has a higher content of biochanin A, whereas, RIMOSTIL has a much higher content of formononetin.

There have been extensive in vitro, in vivo and clinical studies on the efficacy and toxicity of isoflavones (Kuiper, Carlsson et al. 1997; Day, DuPont et al. 1998; Nagel, vom Saal et al. 1998; Pike, Brzozowski et al. 1999; Setchell and Cassidy 1999; Chang, Churchwell et al. 2000; Coldham and Sauer 2000; Izumi, Piskula et al. 2000; Setchell, Brown et al. 2001; Howes, Waring et al. 2002; Liu and Hu 2002; Setchell, Brown et al. 2002; Bowey, Adlercreutz et al. 2003; Setchell, Brown et al. 2003; Setchell, Faughnan et al. 2003; Setchell and Lydeking-Olsen 2003; Atkinson, Compston et al. 2004; Jia, Chen et al. 2004; Schult, Ensrud et al. 2004; Beck, Rohr et al. 2005; Chen, Lin et al. 2005; Chen, Wang et al. 2005; Gu, Laly et al. 2005; Li, Zhang et al. 2005; Setchell, Clerici et al. 2005; Ge, Chen et al. 2006; Kano, Takayanagi et al. 2006; Setchell and Cole 2006; Rachon, Vortherms et al. 2007; Rimoldi, Christoffel et al. 2007; Sepehr, Cooke et al. 2007; Wuttke, Jarry et al. 2007; Rachon, Menche et al. 2008; Wang, Chen et al. 2008). These studies show trends that are pertinent to the design of an optimal red clover product. The absorption of genistein and daidzein is dose dependent, suggesting at higher doses the bioavailability of these moieties decreases (Setchell, Brown et al. 2003; Setchell, Faughnan et al. 2003). The effect of red clover products on osteoporosis is not dependent on dose and ratio of isoflavones in humans (Clifton-Bligh, Baber et al. 2001; Kelly 2002; Schult, Ensrud et al. 2004). However, the toxicity of isoflavones is always associated with a higher concentration or dose (Rachon, Vortherms et al. 2007; Engelhardt and Riedl 2008; Engelhardt and Riedl 2008; Rachon, Menche et al. 2008). The apparent dichotomy between dose and efficacy, and dose and toxicity, may be partly related to tissue specific distribution of isoflavones in the body (Yoshida, Tsukamoto et al. 1985; Kuiper, Carlsson et al. 1997; Chang, Churchwell et al. 2000; Coldham and Sauer 2000; Gu, Laly et al. 2005). For osteoporosis, an understanding of concentration profile in bone is important. Bone isoflavone concentrations are generally not available, except the study reported by Coldham and Sauer (2000) who studied tissue distribution of genistein in rats. According to this study, bone concentration of genistein is among the lowest of all tissues and organs. There is no bone data reported for other isoflavones. Since the product is developed for osteoporosis, the following questions are raised: 1. Is bone distribution dose dependent? 2. Are there any interactions between active and/or inactive moieties in the bone? 3. Are the conjugates found in the bone or do the conjugates act as a reservoir for the aglycones?

Relative absorption of isoflavone glycoside and their respective aglycones is a subject of controversy. Since protocols employed for these studies were not uniform, it is difficult to compare results directly. The bioavailability of genistein and daidzein glycosides was reported to be higher than the aglycones in rats (Sepehr, Cooke et al. 2007) and humans (Setchell, Brown et al. 2001). However, Izumi et al. (2000) showed that soy isoflavone aglycones are absorbed faster and in higher amounts than their glucosides in humans. Similar results were reported by Kano et al. (2006). Studies performed by other groups showed no difference in absorption (Richelle, Pridmore-Merten et al. 2002; Tsunoda, Pomeroy et al. 2002; Zubik and Meydani 2003). Although the cause of controversy is not readily apparent, the low solubility of the aglycones in a preparation may have a profound effect on their dissolution, metabolism and absorption.

Metabolism of isoflavones is mainly mediated by Phase II enzymes in the enterocytes and hepatocytes. Plasma glucuronide and sulfate conjugates concentrations are higher than 95% of the total phytoestrogen content (Howes, Waring et al. 2002). Phase I metabolism also takes place and it has been shown that CYP isozymes are involved in mediating these reactions (Heinonen, Wahala et al. 2004; Chen, Lin et al. 2005). Although metabolism of individual isoflavones in rats has been well characterized (Jia, Chen et al. 2004; Chen, Lin et al. 2005; Chen, Wang et al. 2005), interaction between components has not been evaluated.

Pharmacokinetics of soy isoflavones have been studied both in animals (Sepehr, Cooke et al. 2007) and humans (Howes, Waring et al. 2002; Moon, Sagawa et al. 2006). The bioavailability values of daidzein and genistein are ˜25% and ˜30-40%, respectively (Sepehr, Cooke et al. 2007) in the rat. The most dominant species in plasma are the respective glucuronide conjugates. Aglycones account for less than 5% of total isoflavone concentration in plasma (Setchell, Brown et al. 2001; Moon, Sagawa et al. 2006). The half-life of these components is typically between 8 to 12 hours (Setchell, Brown et al. 2003). The long half-life was used to justify daily dosing of these isoflavones. Plasma protein binding of biochanin A in rats is 1.5% (Moon, Sagawa et al. 2006). There is no other plasma binding data available for other isoflavone aglycones.

The effects of individual isoflavone on bone formation and resorption have been evaluated (Li, Zhang et al. 2005; Ge, Chen et al. 2006). Concentrations higher than 10⁻⁷ molar were found to have a positive effect. The estrogenic effects; however, is in the range of 0.2 μM. A question has been raised in the literature concerning whether humans are properly dosed for the indications of isoflavone because in vitro data show that the concentration requires for activity is way higher than that observed in human tissues (Coldham and Sauer 2000).

Clinical studies show that extracts of red clover or soy are safe; however, their efficacies are also equivocal. Although there are proprietary products in the market, which have shown potentials for treating or preventing postmenopausal osteoporosis, these products, unfortunately do not have the quality of a drug. The major shortcomings for the design of these products in the market are that they have not taken into consideration of the interplay between pharmacokinetics and pharmacodynamics. In other words, proper dosage and/or dosing interval are empirically decided.

### PRIOR ART

A series of patents were issued to Kelly on phytoestrogens and Red clover over the period of 1998 to 2003. In 1998, Kelly developed compositions enriched with phytoestrogens containing Formononetin, Biochanin A, Genistein and Daidzein (Kelly 1998). These compositions could be in the form of food-additives, capsules or tablets for promoting health in cancer, pre-menstrual syndrome, menopause or hypercholesterolemia. The dose range of total phytoestrogens was proposed to be anywhere between 20-200 mg, preferably, the dosage is from 50 to 150 mg. The rationale for coming up with these dosages was based on the daily dietary intake of phytoestrogens in countries, such as Japan, India, South America, North Africa, etc., where there is a high reliance on legumes. There is no attention paid to the utilization of various forms individual phytoestrogens, for example, glucosides vs. aglycone; therefore, the dosage design was not based on the pharmacokinetics of these components and therefore, a dose-, concentration-effect relationship was not established.

In a US patent Kelly (2002) questioned the relationship between estrogenicity of the four major isoflavones: formononetin, biochanin A, daidzein and genistein, which are present in high quantities in Red clover; and daidzein and genistein in soya. Epidemiological studies in populations who consumed a diet containing high leguminous food, inferred that genistein, the isoflavone with the highest estrogenic activity, was the most effective isoflavone. However, according to Kelly this deduction is questionable because Fujita and Fukase (Fujita and Fukase 1992) reported that the bone mass was similar between Japanese and U.S. populations despite the Japanese has a higher quantity of phytoestrogens in their diet. Kelly also cited Tobe's work (Tobe, Komiyama et al. 1997) indicating that daidzein increased bone resorption rate; therefore, the effect of daidzein on osteoporosis was questioned. In the 2002 US patent, Kelly (2002) reported that, despite its insignificant estrogenicity, formononetin was the isoflavone which is more effective for osteoporosis and without the side effects caused by other biologically active plant materials such as coumesterols.

The finding that formononetin was the sole active ingredient was based on the observation that after oral administration, formononetin has measurable plasma levels and the half-life of formononetin was 20 hours, a lot longer than what has been reported in the literature.

Like the other three isoflavones, it is well known that formononetin undergoes extensive first-pass intestinal (Chen, Lin et al. 2003) and hepatic metabolism (Tolleson, Doerge et al. 2002), the oral bioavailability of formononetin is likely to be very low (Baillard, Bianchi et al. 2007). In addition to Phase II metabolism, formononetin is metabolized in the gut and the liver to produce daidzein; the plasma level of conjugated daidzein after the administration of a mixture containing significant amount of formononetin is a lot higher than that of formononetin (Howes, Waring et al. 2002). The area under the plasma concentration vs. time curve values of formononetin was approximately one seventh of that of daidzein. Although Howes et al. (2002) reported similar half-life values for formononetin; this phytoestrogen certainly was not the dominant species in the blood. In his patent, Kelly (2002) did not reveal any evidence to suggest that formononetin was the most important active ingredient. One should also note that the plasma levels of these isoflavones are actually present in the conjugated forms. This is often mistaken as the unmetabolized form of the aglycone. The actual level of the intact aglycone is a lower than that of the conjugates.

In all of the clinical studies cited in the patent (Kelly 2002), other than the “discovery” of the long half-life of formononetin, there was no convincing data to substantiate that formononetin was the only active moiety. It should be pointed out that the half-life values of the other isoflavones are very similar, making Kelly's (2002) half-life argument unconvincing (Howes, Waring et al. 2002).

In a review article, Booth et al. (2006) evaluated five clinical trials. The main aim of these trials was to study the effects of Red clover on osteoporosis. Two trials used extracts containing high formononetin (Kelly 2000; Clifton-Bligh, Baber et al. 2001). One trial (Atkinson, Compston et al. 2004) employed PROMENSIL®, a product contained a higher proportion of biochanin A. In another trial, the effects of PROMENSIL® and RIMOSTIL (Schult, Ensrud et al. 2004) were compared. In yet another trial (Hale, Hughes et al. 2001), the ratio of formononetin and biochanin was not specified.

Results in these trials showed that an extract containing a high ratio of formononetin to the other isoflavones has significant effects on increasing the cortical bone density, but it has insignificant effects on the trabecular bone density (Kelly 2000; Clifton-Bligh, Baber et al. 2001). However, when bone turnover markers were employed, insignificant differences were found between study medications and placebo (Hale, Hughes et al. 2001; Atkinson, Compston et al. 2004; Schult, Ensrud et al. 2004), regardless of the formononetin and biochanin A ratios. Booth (2006) suggested that the bone turnover markers might be unreliable. The clinical trial results reported by Kelly (2002) contradicts to that of Booth (2006). Both the bone turnover markers and bone mineral density changed significantly after Red clover treatment. Due the small sample size, Kelly's claims in his inventions are not clinically substantiated (Kelly 2000; Kelly 2002).

Despite the claims cited in Kelly's inventions, total isoflavone dosages lower than 25 mg have not been shown to be clinically effective towards osteoporosis as cited in Booth's review (Booth, Piersen et al. 2006).

Kelly (2002) has made a broad claim on the dosage forms for different routes of administration. He obviously did not recognize the potential difference in clinical response that these products could make. To highlight the therapeutic effect of formononetin, one should not administer this isoflavone through a route that would be converted mostly to other metabolites such as daidzein and equol. There is also no evidence in Kelly's patent (2002) that in the design of dosage forms; the interplay between solubility, first-pass gut and liver metabolism was taken into consideration. These factors, as reported in this invention, are the most important in the design of an optimal dosage form. On the contrary, Kelly (2003) cited that metabolism did not occur after phytoestrogens and their metabolites are absorbed from the gut. Obviously, this citation is erred. There are plenty of evidences to suggest that metabolism in the enterocyte and liver occurs (Howes, Waring et al. 2002; Tolleson, Doerge et al. 2002; Jia, Chen et al. 2004; Chen, Wang et al. 2005).

There is evidence in the literature including patents and products in the market to show that the major phytoestrogens of Red clover: formononetin and biochanin A and some of their Phase I metabolites, such as daidzein and genistein are active in reducing bone loss (Kelly 1998; Kelly 2000; Kelly 2002; Migliaccio and Anderson 2003; Cassidy, Albertazzi et al. 2006). The source of phytoestrogens in Red clover mainly comes from the glucosides. There is no consensus in the literature concerning the relative effectiveness of the glucosides and their respective aglycones. There have been attempts to define an optimal ratio for the phytoestrogens; however, no clear-cut answer has been provided scientifically and clinically.

Judging from the body of knowledge on Red clover, there is no information on how the phytoestrogens work together pharmacokinetically and pharmacodynamically. In this invention, the interplay between these “active” components was evaluated in detail using PPT. Based on the data generated, the disadvantages of the current products in the market have been unveiled. A new product with definitive proportions of phytoestrogens is defined. The dosage of the new product is a small fraction of those available in the market. The advantage of this product is its consistency. By modifying the mode of delivery, the other advantage of this product is the increase in the absorption of the aglycones and their rate of conversion to their respective bioactives metabolites.

### DETAILED DESCRIPTION OF THE FIGURES

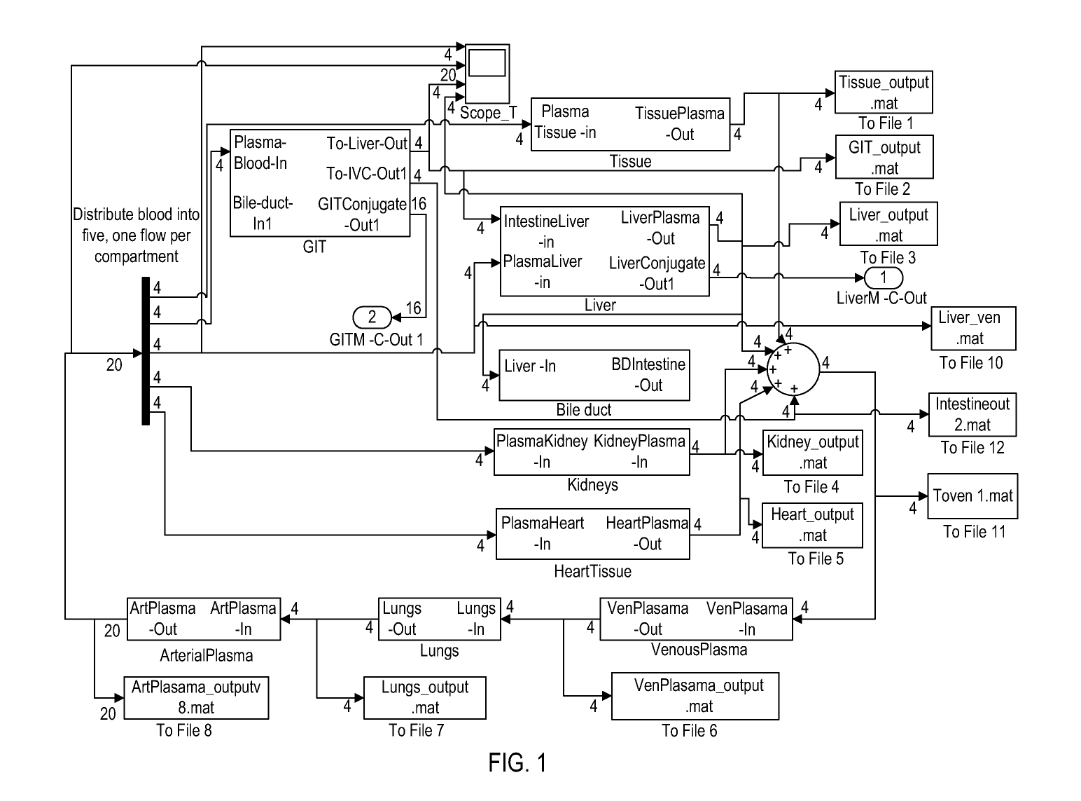

FIG. 1 shows the complete PBPK algorithm constructed using the circulatory system. It includes the gastrointestinal tract (GIT), tissue, liver, bile duct, kidney, heart tissue, venous plasma, lung and the arterial plasma compartment.

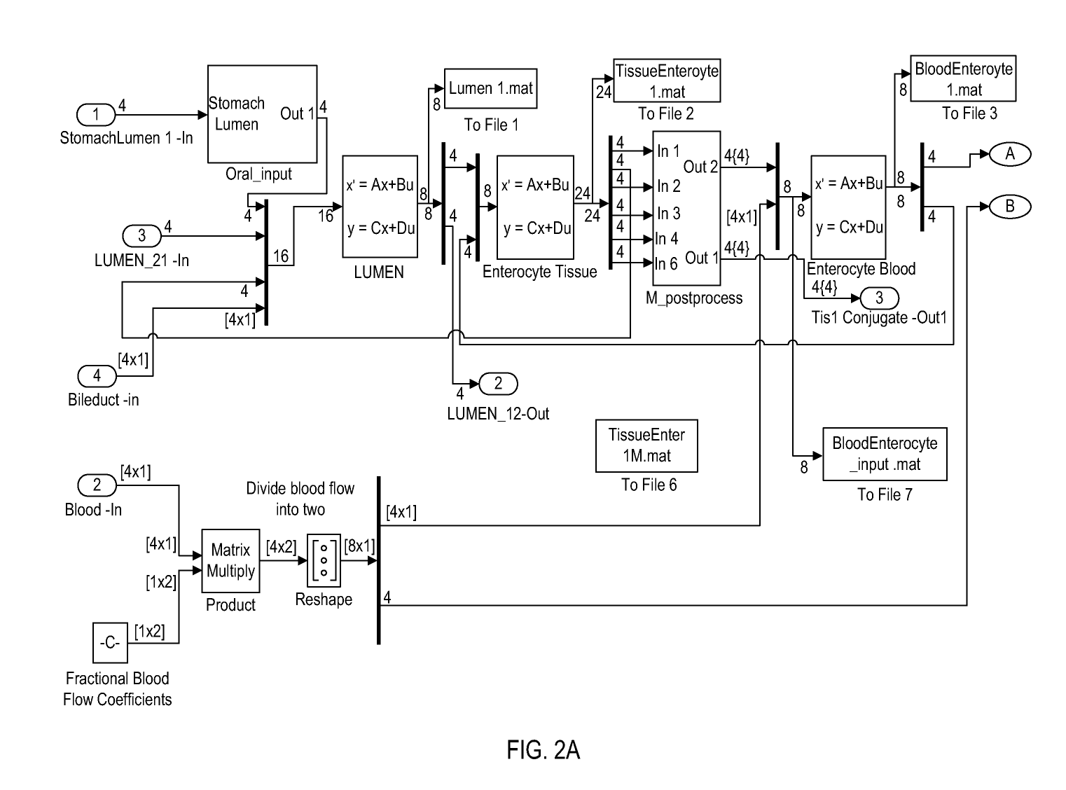

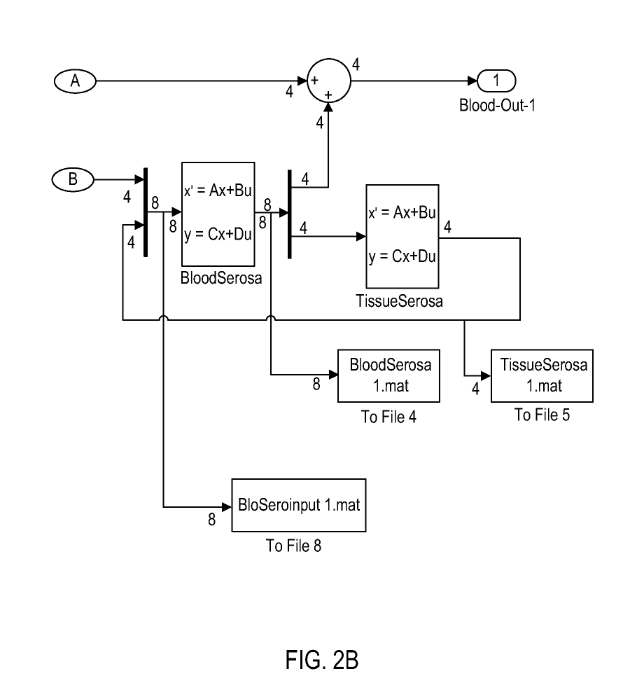

FIG. 2A shows the duodenal compartment architecture for enterocytes. The M_process compartment simulates metabolic processes.

FIG. 2B shows the duodenal compartment architecture for serosa and outputs.

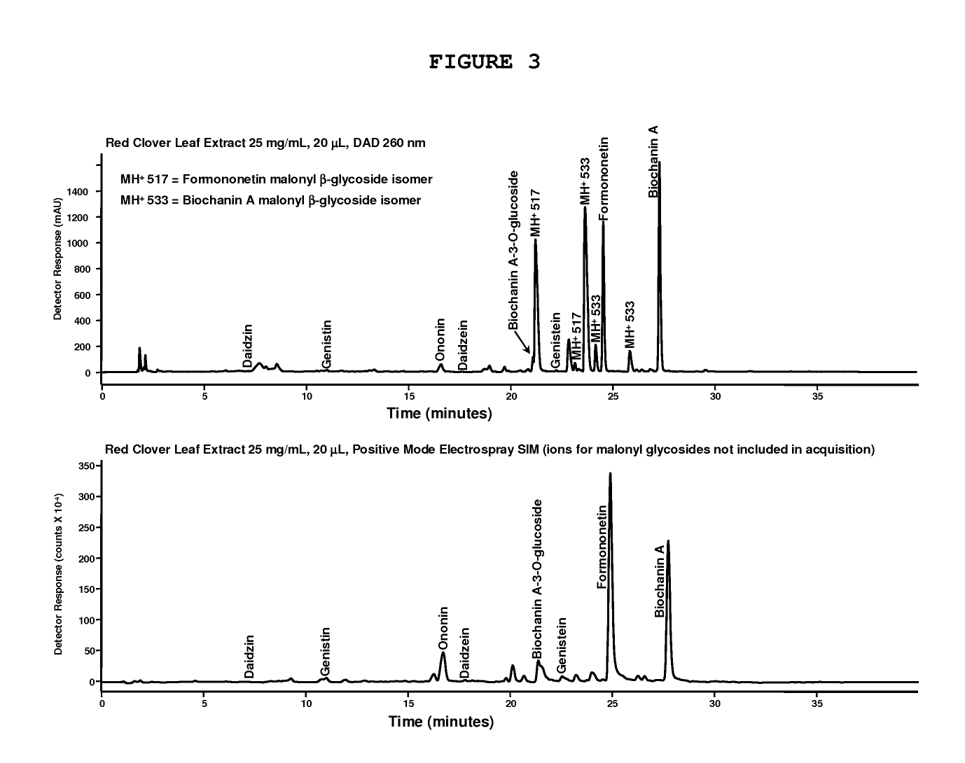

FIG. 3 shows a typical LC/MS chromatogram showing the composition of a Red clover extract.

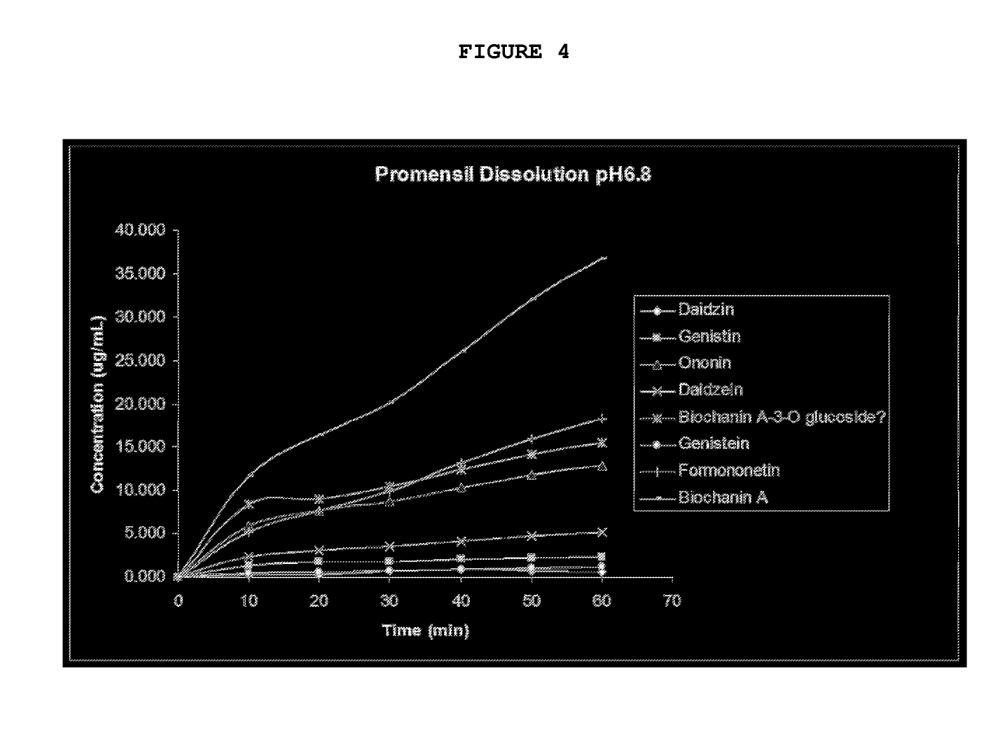

FIG. 4 shows the dissolution profile of Promensil at pH 6.8. The dissolution medium, containing sodium lauryl sulfate, is designed to evaluate the dissolution of low solubility substances.

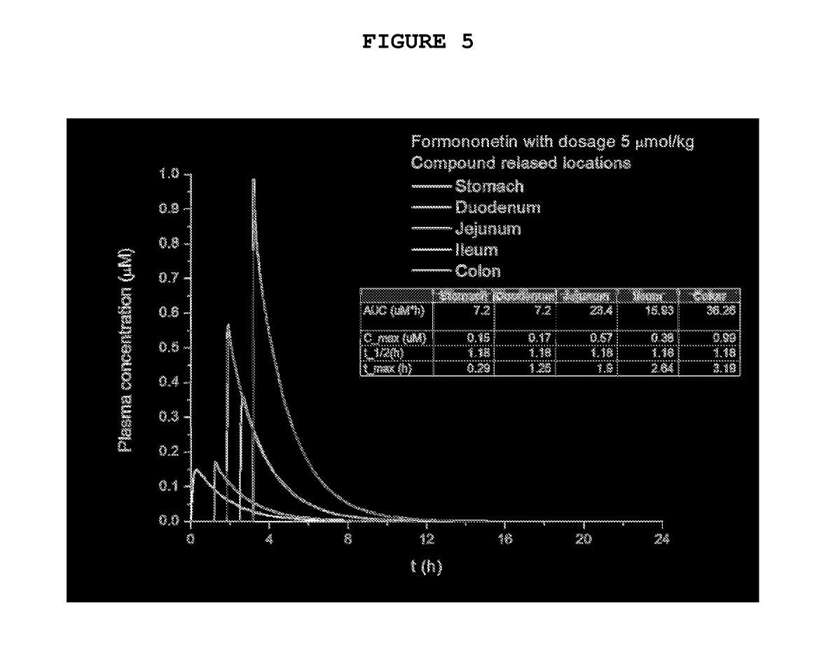

FIG. 5 shows the plasma concentrations of pure formononetin released at different locations.

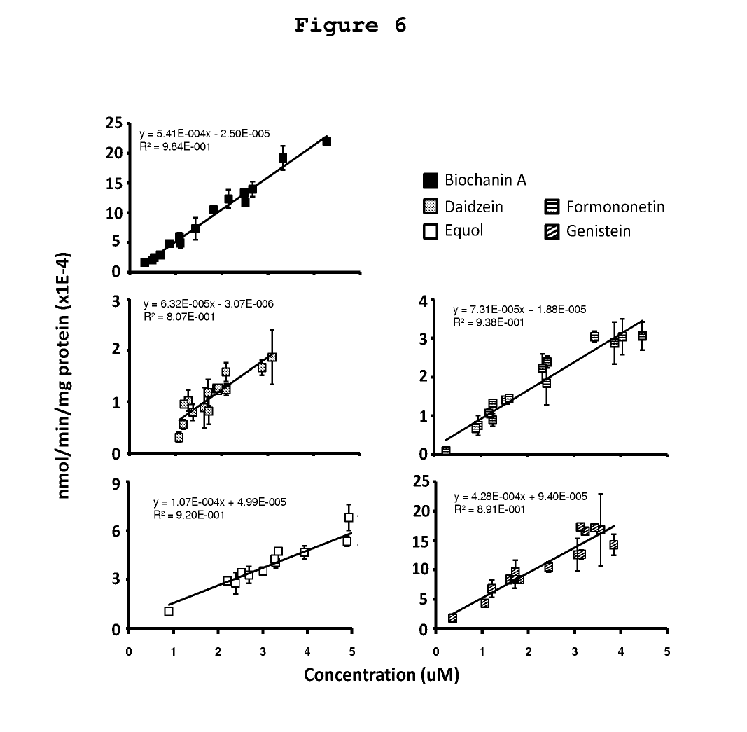

FIG. 6 shows the metabolism of the isoflavone mixtures by human intestinal microsomes.

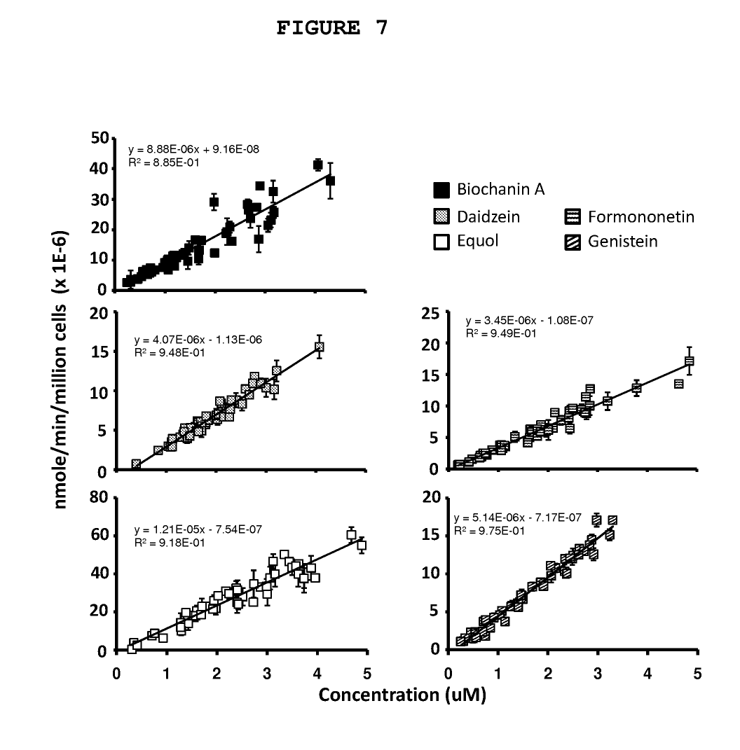

FIG. 7 shows the metabolism of the isoflavone mixtures by human hepatocytes.

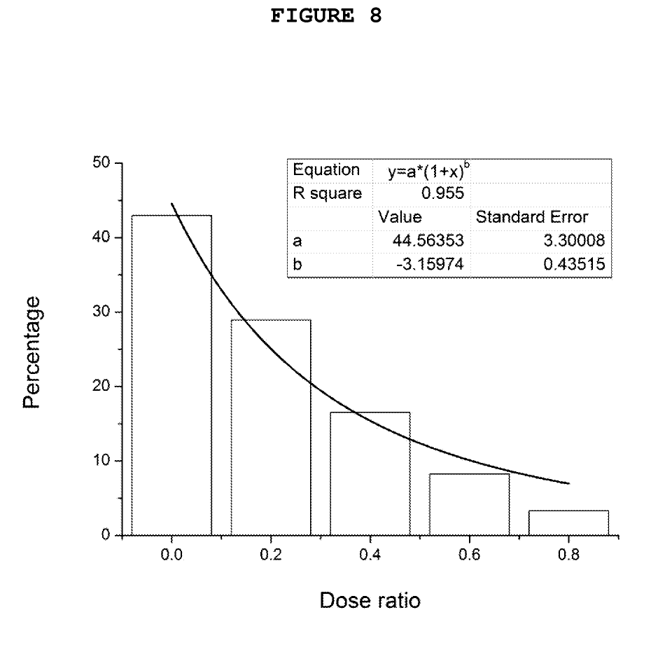

FIG. 8 shows a Histogram of dose ratio distribution for individual isoflavones in the 121 mixtures. The solid line shows the fitted power law distribution and parameters are shown in the inset.

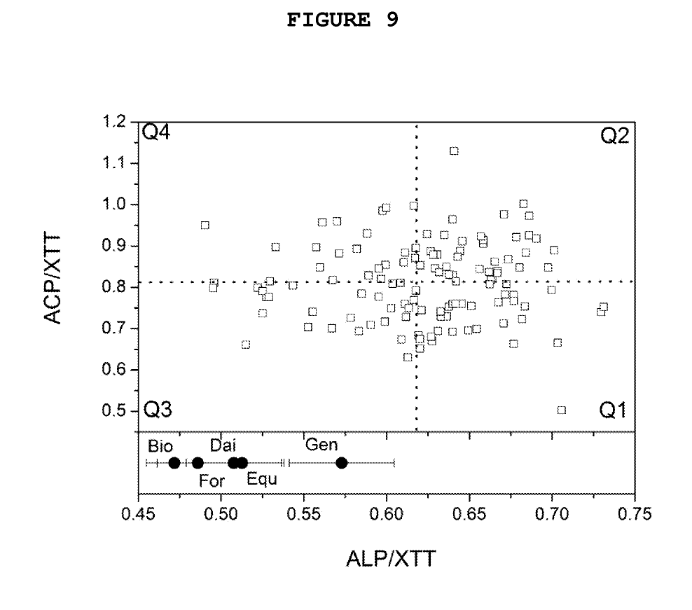

FIG. 9 shows the ability for the 121 combinations (open square) to form osteoblast (ALP/XTT) and inhibit osteoclast formation (ACP/XTT) is shown on the upper panel. The lower panel shows osteoblast forming activity (mean±SE) of pure isoflavone measured at 1 μM, where “Bio” stands for Biochanin A, “For” is formononetin, “Dai” is daidzein, “Equ” is equol and “Gen” is genistein.

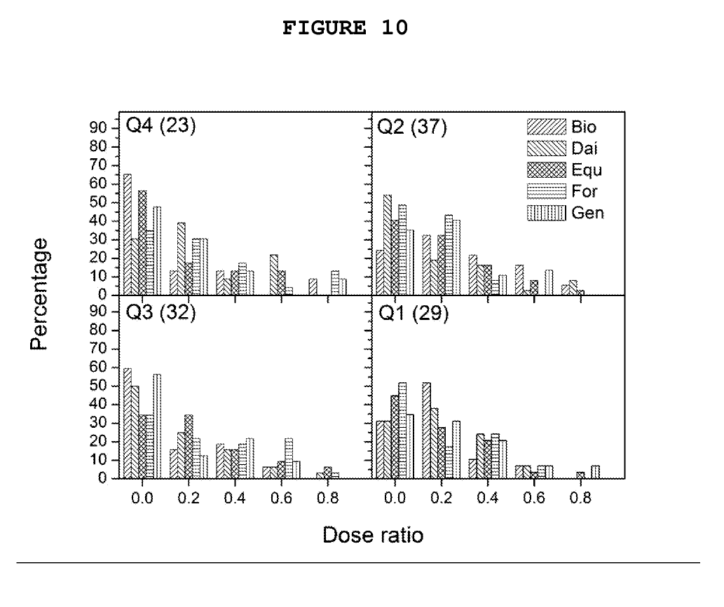

FIG. 10 shows a histogram of five isoflavones dose ratio in four quadrants defined in FIG. 2. The number inside round bracket is the number of data points in this quadrant. The abbreviations in the legend are defined in FIG. 9.

### SUMMARY OF THE INVENTION

The present invention discloses a process of developing optimized red clover products using pharmaceutical platform technology. In one embodiment, the product is a pH sensitive release dosage form containing 5 to 50 mg of red clover extract. In another embodiment, the product is an immediate release dosage form containing 5 to 50 mg of Red clover extract. In another embodiment, the product consists of a blend of the immediate and pH sensitive release dosage form containing 5 to 50 mg of Red clover extract. In another embodiment, the extract has at least 30% of formononetin.

### DETAILED DESCRIPTION OF THE INVENTION

The present invention describes a method of using the pharmaceutical platform technology (Tam and Tuszynski 2008) to design a product using red clover extract which will consist of a proper dosage of formononetin and biochanin A in such a way that it would deliver adequate amounts of these phytoestrogens to a biophase such as bone. In one embodiment, by using an immediate-release delivery system, it is expected that the bioavailability of these two phytoestrogens be optimized.

In another embodiment, by using a sustained-release dosage form, the ratio of the active metabolites such as daidzein, equol and genistein, can be optimized.

In another embodiment, by using a combination of immediate- and sustained-release dosage form, the plasma levels of the precursors and active metabolites can be adjusted, depending on the relative potencies of the product.

The major application of PPT is to predict individual component time course after a mixture is administered (Tam and Tuszynski 2008). In the present invention, a mixture is an extract of red clover. The backbone of PPT is the construction of a set of physiologically based pharmacokinetic and pharmacodynamic models for describing the time course of concentration and effect in different animal species including human. Parameters describing these models are derived from physiological data for a specific animal species and kinetic and activity parameters are derived from in vitro studies. Physiological parameters include but not limited to gastric emptying and intestinal transit time, hepatic blood and cardiac blood flow rate, etc. Component specific parameters include but not limited to intestinal permeability, rate of intestinal and hepatic metabolism, plasma protein binding, tissue distribution and dose-response relationship. The unique feature of PPT is its ability to estimate pharmacokinetic and pharmacodynamic activity of individual components and interaction between these components in a mixture without purification of these individual components.

As shown later, the two major aglycones of Red clover are highly insoluble in the gastrointestinal fluids. Regardless of the ratio of the two, relative solubility, absorption and metabolism by the intestinal flora will dictate the consistency of the blood levels of these components and their metabolites. In one embodiment, an immediate-release dosage form is designed to release the two isoflavones: formononetin and biochanin A in the stomach. The intention is to promote solubility and absorption of the two isoflavones. Thus, the absorption of the two components will be more consistent. This dosage form is designed to promote absorption and to minimize metabolism by the intestinal flora. This dosage form will be useful for preparations, which are indicated for the prevention of osteoporosis and particularly for the prevention of cancer.

A timed-release dosage form is designed to release a drug in the lower portion of the intestine. The intention is to expose the major isoflavones: formononetin and biochanin A to intestinal flora to promote the conversion to the bioactive metabolites: daidzein, equol and genistein. These metabolites have been shown to be bioactive and their potencies on cell differentiation are fairly similar. In one embodiment, the phytoestrogens are solubilized in a vegetable oil to promote dissolution of the phytoestrogens. In another embodiment, the oil containing the dissolved phytoestrogens is adsorbed onto silica to form granules. In another embodiment, the granules are coated with a timed-release coating such as but not limited to shellac, which will release these granules in jejunum or ileum (Pearnchob and Bodmeier 2003; Pearnchob, Siepmann et al. 2003; Pearnchob, Dashevsky et al. 2004; Former, Theurer et al. 2006).

In one embodiment, a dosage form consisting of the immediate-release and time-release components is designed. The purpose of this design is to provide and optimal ratios of formononetin, biochanin A, daidzein and genistein in order to achieve optimal efficacy.

In one embodiment, the present invention provides a method of identifying a composition for treating or preventing osteoporosis, comprising the steps of a. obtaining a red clover (Beck, Unterrieder et al.) extract comprising a plurality of components which are the aglycone forms of phytoestrogens; b. determining parameters describing the rate of metabolism of the components in a plurality of mammalian tissue systems; c. determining parameters describing distribution of the components in a plurality of mammalian tissue systems; and d. inputting the parameters into in silico models which will generate outputs to predict the pharmacokinetics and pharmacodynamics properties of the components in vivo, thereby providing a composition comprising the components useful for treatment or prevention of osteoporosis. In general, the parameters for the above methods are obtained from in vitro or in vivo studies. Representative examples of mammalian tissue systems include, but are not limited to, gastrointestinal tract, liver, kidney, blood, mammary gland, uterus, prostate, brain, and bone.

In one embodiment, rate of degradation (dc/dt) is generally assumed to be first order. What this means is that the rate of decomposition is concentration dependent:

$$\frac{d c}{d t} = {C}_{0} {e}^{- Kt}$$

Where c is concentration at time t, C₀ is the concentration at time zero and K is the first order degradation rate constant. This rate equation can be integrated and transformed to:

C=C₀e^{-Kt}

The half-life of a substance is determined as time for 50% of the original concentration to disappear. From the above equation, half-life, t_{1/2}, is defined as:

$${t}_{1 / 2} = \frac{0.693}{K}$$

In another embodiment, the method of the present invention further comprises the step of determining parameters for active metabolites of the components of the extract according to steps a through d described above, wherein results of the above determination will predict the pharmacokinetics and pharmacodynamics properties of the components and their metabolites in vivo. In general, pharmacokinetics and pharmacodynamics properties comprise concentration-time profiles and response-time profiles for the components and their metabolites.

In one embodiment, the method of the present invention comprises mathematical models that are capable of solving multiple unknowns which are linearly independent or interacting with each other. For example, the models include a model of weighted linear functions and the same model with added higher-order polynomial terms in single component doses and terms in the products of pairs of doses. In one embodiment, the mathematical models comprise

$$r \approx \overline{r} + {\sum }_{i}^{ } {w}_{i} ( {d}_{i} - {\overline{d}}_{i} ) + {\sum }_{i}^{ } {{w}_{i}^{′} ( {d}_{i} - {\overline{d}}_{i} )}^{2} + {\sum }_{i , j}^{ } {w}_{i , j} ( {d}_{i} - {\overline{d}}_{i} ) ( {d}_{j} - {\overline{d}}_{j} ) ,$$

wherein r is linearized response, r̅ is the average linearized response; wᵢ is weight of the i component (relates to potency), dᵢ is the dose of component i and d̅ᵢ and d̅ⱼ are average dose of the i^{th} and j^{th} component, w_{i,j} is the weight of the interacting pair.

In another embodiment, the mathematical models comprise

$$A = {α}_{0} + {\sum }_{i = 1}^{n} {α}_{i} {x}_{i} + {\sum }_{i = 1}^{n} {\sum }_{j = 1}^{n} {β}_{i , j} {x}_{i} {x}_{j} ,$$

wherein α₀ and αᵢ are baseline activity and activity coefficient of component i respectively, xᵢ and xⱼ are components i and j respectively, β_{i,j} is the activity coefficient of the interacting pair, xᵢ and xⱼ, wherein said equation is able to predict an optimized composition of the extract to achieve maximum possible potency.

In yet another embodiment, examples of applicable mathematical models include, but are not limited to, least absolute shrinkage and selection operator (LASSO), wavelet-based deconvolution, compressed sensing, and gradient projection algorithm.

In another embodiment, entropic component analysis consisting of: (a) assigning probabilistic models to all possible combinations of variables and (b) determining the ranking scheme of these models.

In one embodiment, determining the rate of metabolism in gastrointestinal tract comprises in vitro assays. For example, such assays comprise artificial gastric or intestinal juice, intestinal flora, intestinal microsomes, or permeability studies using cultured cells or intestinal tissues (e.g. Caco-2 cells or MDCK cells).

In one embodiment, determining the rate of metabolism in liver comprises assays using freshly harvested hepatocytes, cryopreserved hepatocytes, hepatic microsomes, hepatic cytosol or S-9 fractions.

In one embodiment, the determination of distribution in blood or plasma comprises determining binding to plasma protein, binding to blood protein, pKa, log P, log D, and volume of distribution of a component.

The present invention also provides a red clover composition comprising multiple components as identified by the method disclosed herein, wherein the components have desirable in vivo pharmacokinetics and pharmacodynamics properties as determined by the method disclosed herein. In one embodiment, the Red clover comprises formononetin and biochanin A in amounts determined by the method disclosed herein. For example, the formononetin and biochanin A may have a ratio ranging from 5:0 to 1:5. In another embodiment, the red clover composition may be formulated in a dosage ranging from 5 to 40 mg of formononetin. In another embodiment, the red clover composition is formulated in a timed or controlled released dosage form. In yet another embodiment, an immediate release dosage form is formulated. In yet another embodiment, a dosage form containing immediate and time-release dosage form is designed. In yet another embodiment, the red clover composition is formulated in a form for increased solubility and absorption of formononetin and biochanin A. In another embodiment, the plasma ratio of the two aglycones and their metabolites can be adjusted by preparing a dosage form, which contains an immediate release and a time-release component.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

The objective of this example is to establish a physiologically based pharmacokinetic/pharmacodynamic (PBPKPD) model to describe the time course of concentration and effects of the bioactives and their metabolites in the body. The pharmacodynamic model is setup to describe the rate of bone formation and resorption.

A basic PBPK model for a single component was used as a starting point for the construction of the multiple component PBPKPD model. The concept of the multiple component PBPKPD model has been described in the patent application by Tam and Tuszynski (Tam and Tuszynski 2008).

The main phytoestrogens in Red clover are metabolized in the gut lumen, gut wall and the liver. Some of the Phase II metabolites of these phytoestrogens are excreted into the bile; therefore, biliary excretion of phytoestrogens into the intestinal lumen and enterohepatic recirculation is incorporated into the basic model.

The distribution of phytoestrogens is affected by transporters lining the intestinal tract and hepatocytes; active uptake and excretion by these transporters are also included into the basic PBPKPD model.

In order to simulate the circulation of parent drugs and corresponding metabolites simultaneously, the basic model was expanded to accommodate the pharmacokinetics of the components of red clover and their metabolites. According to our data and the data reported in the literature, metabolism of Red clover phytoestrogens occurs at enterocytes along the intestine and liver. Based on this information, the circulatory system was constructed to account for pharmacokinetics of the parent drugs (phytoestrogens) and their respective metabolites (FIG. 1). Additional compartments in the intestine and liver were included to describe metabolic processes. As an example, FIG. 2 shows the pathway in duodenum. The M_process box simulates metabolic processes. The product of these metabolic compartments will be transported from the circulatory system for parent drugs to the other circulatory system as shown in FIG. 1.

### Example 2

The objective of this study is to study the events that occur in the lumen of the gastrointestinal tract. The goals are to identify the stability of Red clover components, their physical and enzymatic stability, the rate of solution and the absorbability the components and their metabolites.

Twenty five red clover extracts containing a diverse composition of biochanin A, formononetin, genistein, daidzein and their glucosides, along with other minute quantities of coumestrol and lignans have been prepared either using solvent extraction or a variety of cultivars. In one embodiment, the aerial portion of red clovers, leaves, stems or leaves and stems, were dried powdered. The plant material was extracted with 50% ethanol at 50° C. for 1 hour. The resultant sample was centrifuged and the ethanolic component was removed and dried.

A chromatographic analysis showed that the major ingredients in these extracts are the glucosides of formononetin and biochanin A and their respective aglycones. Tiny amounts of genistein, daidzein and their glycosides were also found. These data are consistent with what is reported in the literature (FIG. 3, (Krenn, 2002 #687)).

A study of the stability of the key components of a Red clover extract in artificial gastric and intestinal juice showed that the glucosides were partially (<25%) converted to their respective aglycones.

According to the literature, formononetin and biochanin A are demethylated by the intestinal micro flora to produce two active metabolites daidzein and genistein, respectively (Hur and Rafii 2000). However, the importance of this metabolic pathway is questioned (Tolleson, Doerge et al. 2002). To understand the relative importance of fecal metabolism, the metabolic rate of red clover phytoestrogens will be measured by subjecting Red clover extracts to incubation with human and rat feces under anaerobic conditions (Rufer, Maul et al. 2007). These extracts will be incubated for various durations to produce a diverse profile of metabolites and precursors. It is anticipated that the major metabolites are derived from formononetin and biochanin A. These metabolites include but not limited to daidzein and its metabolite, equol, and genistein.

Red clover extracts were subjected to permeability measurements using CaCO₂ and MDCK cells. Permeability across these barriers provides an indication of absorbability.

The permeability values of formononetin, biochanin A, daidzein, genistein and equol are quite high, suggesting that these components are highly absorbable (Table 2). However, the glucosides of the aglycones such as biochanin A glucoside and ononin have poor permeability suggesting the bioavailability of the sugar conjugates are poorly absorbed. These results are consistent with that reported in the literature in that when these glycosides are administered to either animals or humans, no glycosides could be detected in the blood stream (Setchell, Brown et al. 2002).

TABLE 2
CaCo-2 permeability of isoflavone in a Red clover extract

| Isoflavones | Mean Peff, cm/sec | STDEV |
|---|---|---|
| Biochanin A glucoside | 1.64E−08 | 1.11E−09 |
| Biochanin A | 1.08E−05 | 3.83E−07 |
| Daidzein | 2.66E−05 | 1.11E−06 |
| Daidzin | 5.36E−07 | 8.30E−08 |
| Formononetin | 2.20E−05 | 6.85E−07 |
| Genistein | 2.75E−05 | 1.22E−06 |
| Genistin | 3.46E−07 | 9.20E−08 |
| Ononin | 1.22E−07 | 1.93E−08 |

The results from the permeability study show that it would be beneficial to convert all the glucosides to their respective aglycones. Two advantages of adopting this strategy: a. the variability in the rate and extent of conversion from glucosides to aglycones between subjects will be removed. A more consistent pattern of aglycone absorption is anticipated. b. dosage calculation for the bioactives will be reduced to the aglycones only. This simplifies the standardization process.

An optimal extract of Red clover should consist of the aglycones only. An enzymatic or chemical conversion of the glucosides to their respective aglycones prior to extraction will be desirable.

The results of these studies show that the absorbable components of Red clover extract will be mainly, formononetin, biochanin A, daidzein and genistein. There may be trace amount of natural substances such as that of coumestans and lignans which are not present at measurable quantities and a number of metabolites which was formed as a result of bacterial metabolism.

The solubility of the four major phytoestrogens: formononetin and biochanin A, daidzein and genistein was measured in artificial gastric and intestinal juice. The solubility of these four compounds, in general, is low. Formononetin has the lowest solubility, which is approximately 4 μg/ml at 37° C. in artificial intestinal juice. Biochanin A has a higher solubility, ˜23 μg/ml. The solubility of daidzein and genistein is quite a bit higher, ranging between 80 to 100 μM in buffer at 37° C. This set of data suggests that solubility instead of absorption is a huge issue in terms of bioavailability.

We tested the hypothesis that solubility may be an issue in phytoestrogen absorption. The hypothesis was tested by examining the dissolution profile of a commercially available Red clover product, PROMENSIL® (30 tablets in a box, Lot # [B] 48449, Exp. 03/2011). FIG. 4 shows that the dissolution of phytoestrogens in the product is not complete, lending evidence to support the idea that an inappropriately formulated product will perform erratically because of absorption issues. It should also be pointed out that the phytoestrogens in PROMENSIL® consist of both aglycones and their glucosides. Compounding the bioavailability issue, both of these species are not completely dissolved under the experimental condition studied.

### Example 3

The objective of this example is to evaluate the effects of first-pass gut metabolism on the bioavailability of the major phytoestrogens. The permeability data produced as described in Example 2 and the regional difference in the metabolism of biochanin A and formononetin published by Jia et al. (Jia, Chen et al. 2004) are used to estimate regional bioavailability. By incorporating of these data into the in silico model described in Example 1, administration of formononetin in different regions of the intestine show significantly different results (FIG. 5). The estimated bioavailability of formononetin is five times higher when it is administered directly to colon as compared to that of oral. Similar observations are expected for biochanin A.

The simulation result is consistent with that reported in the literature. Wang et al., (2006) showed that the absorption of formononetin and biochanin A is the highest in the colon when a perfused intestine model was used. The excretion of the glucuronides was also found to be the lowest. The low excretion rate was related to a slower conjugation rate of formononetin in colonic microsomes.

The solubility and first-pass intestinal metabolism issues are likely the major causes of variability of clinical response of Red clover products. The lack of dose-response relationship is also consistent with the solubility issue of the phytoestrogens. The dissolution results obtained from the most studied commercial product, PROMENSIL®, are consistent with this speculation. First pass metabolism is one of the causes of low bioavailability and high variability of drug exposure. For Red clover, the first-pass issue can be minimized if the bioactives can be released at the lower part of the intestine. The low solubility, extensive first-pass gut metabolism and metabolic conversion to its active metabolites, daidzein and equol could explain why formononetin did not out perform other combination of bioactives as that claimed by Kelly in his patent (2002).

### Example 4

During our studies, it was discovered that using extracts for metabolism and efficacy studies may not be appropriate. The reason is that unabsorbable components may have effects on the metabolism and binding to receptors of potential bioactives. Non-absorbable anti-oxidants such as high molecular weight catechins found in grape seed extracts are good examples. Therefore, we feel that it is imperative to use absorbable fractions for our studies. A method for preparing absorbable fractions has been reported and used in this invention (Tam, Lin et al. 2011).

The objectives of this example are to evaluate the metabolism of absorbable components of Red clover extracts.

Human liver microsomes, intestinal microsomes, and hepatocytes were purchased from XenoTech. All chemicals were purchased from Sigma-Aldrich. Isoflavones (biochanin A, daidzein, equol, formononetin, and genistein) were first dissolved in DMSO and then mixed according the ratio listed in Table 3. The final DMSO in buffer or media was kept at 0.1%. For glucuronidation with microsomal incubation, the final reaction mix includes 0.1M phosphate buffer (pH7.4), 5 mM UDPGA, 50 μg alamethicin/mg microsomal protein, 1 mM MgCl₂, 0.5 mg protein/ml. Incubation time was decided through preliminary trials to aim at the disappearance of 50% of isoflavones. To stop the reaction, equal volume of 50% acetonitrile and 50% methanol was added to the reaction mix. Samples were analyzed with LC/MS.

TABLE 3
Isoflavone Mixtures

| | Isoflavone relative ratio | | | | |
|---|---|---|---|---|---|
| | Biochanin A | Daidzein | Equol | Formononetin | Genistein |
| RC1 | 2.22 | 1.53 | 4.08 | 1.67 | 0.50 |
| RC2 | 4.08 | 2.04 | 1.26 | 2.11 | 0.50 |
| RC3 | 0.50 | 3.53 | 3.59 | 2.16 | 0.22 |
| RC4 | 1.60 | 2.41 | 5.60 | 0.10 | 0.29 |
| RC5 | 0.64 | 2.45 | 2.76 | 2.45 | 1.70 |
| RC6 | 2.22 | 1.20 | 3.02 | 0.77 | 2.79 |
| RC7 | 0.90 | 1.81 | 3.07 | 3.83 | 0.40 |
| RC8 | 3.02 | 2.52 | 1.58 | 1.42 | 1.45 |
| RC9 | 1.04 | 1.73 | 1.74 | 2.78 | 2.71 |
| RC10 | 2.37 | 1.39 | 2.99 | 1.96 | 1.29 |
| RC11 | 2.63 | 2.45 | 1.54 | 1.74 | 1.64 |
| RC12 | 1.01 | 1.47 | 2.29 | 1.12 | 4.11 |
| RC13 | 1.55 | 1.80 | 1.36 | 1.81 | 3.47 |
| RC14 | 1.13 | 3.35 | 1.56 | 0.67 | 3.29 |
| RC15 | 4.46 | 2.00 | 0.51 | 1.17 | 1.86 |
| RC16 | 2.49 | 1.10 | 2.52 | 2.97 | 0.93 |
| RC17 | 0.71 | 1.78 | 1.91 | 2.55 | 3.05 |
| RC18 | 0.41 | 1.25 | 3.80 | 0.14 | 4.41 |
| RC19 | 2.93 | 1.96 | 2.32 | 0.95 | 1.84 |
| RC20 | 3.50 | 1.99 | 1.89 | 0.84 | 1.78 |
| RC21 | 2.04 | 2.22 | 1.30 | 1.20 | 3.23 |
| RC22 | 0.14 | 3.24 | 3.34 | 2.92 | 0.36 |
| RC23 | 1.23 | 1.57 | 3.18 | 0.64 | 3.38 |
| RC24 | 0.39 | 2.38 | 1.80 | 2.83 | 2.60 |
| RC25 | 2.99 | 2.94 | 1.10 | 2.31 | 0.65 |
| RC26 | 0.11 | 2.80 | 1.88 | 1.80 | 3.40 |
| RC27 | 1.76 | 1.78 | 2.48 | 2.32 | 1.66 |
| RC28 | 1.00 | 1.31 | 4.85 | 0.16 | 2.68 |
| RC29 | 0.59 | 3.46 | 2.52 | 1.77 | 1.66 |
| RC30 | 0.43 | 4.95 | 0.31 | 0.52 | 3.79 |
| RC31 | 0.37 | 3.14 | 3.14 | 2.77 | 0.58 |
| RC32 | 1.83 | 1.44 | 2.70 | 1.80 | 2.22 |
| RC33 | 2.35 | 2.24 | 4.28 | 0.43 | 0.69 |
| RC34 | 0.77 | 1.73 | 3.68 | 3.56 | 0.27 |
| RC35 | 1.52 | 2.01 | 1.59 | 2.50 | 2.39 |
| RC36 | 1.54 | 2.28 | 0.08 | 5.21 | 0.88 |
| RC37 | 0.71 | 2.47 | 1.32 | 3.25 | 2.25 |
| RC38 | 3.25 | 3.14 | 0.18 | 2.52 | 0.92 |
| RC39 | 1.28 | 1.65 | 2.84 | 1.26 | 2.97 |
| RC40 | 1.04 | 2.41 | 2.29 | 1.86 | 2.40 |
| RC41 | 3.11 | 0.83 | 0.60 | 4.66 | 0.80 |
| RC42 | 0.14 | 2.40 | 3.78 | 2.87 | 0.82 |
| RC43 | 1.31 | 1.63 | 3.48 | 0.55 | 3.03 |
| RC44 | 3.04 | 1.77 | 0.90 | 2.02 | 2.27 |
| RC45 | 0.63 | 3.10 | 1.19 | 2.02 | 3.06 |
| RC46 | 1.12 | 1.29 | 2.74 | 1.18 | 3.67 |
| RC47 | 3.58 | 2.66 | 1.25 | 2.12 | 0.39 |
| RC48 | 2.62 | 2.54 | 2.36 | 0.75 | 1.72 |
| RC49 | 0.20 | 3.86 | 2.84 | 1.47 | 1.62 |
| RC50 | 1.85 | 0.41 | 2.62 | 2.06 | 3.05 |
| RC51 | 3.98 | 3.63 | 0.19 | 0.39 | 1.82 |
| RC52 | 1.70 | 2.09 | 1.30 | 2.62 | 2.29 |
| RC53 | 3.81 | 2.09 | 1.28 | 0.42 | 2.40 |
| RC54 | 4.55 | 2.47 | 0.53 | 1.56 | 0.90 |
| RC55 | 1.09 | 1.71 | 2.04 | 1.77 | 3.39 |
| RC56 | 1.57 | 2.86 | 1.93 | 2.90 | 0.73 |
| RC57 | 2.82 | 1.20 | 2.80 | 2.90 | 0.28 |
| RC58 | 1.09 | 0.96 | 2.86 | 3.62 | 1.47 |
| RC59 | 3.18 | 2.75 | 0.03 | 2.46 | 1.58 |
| RC60 | 4.04 | 0.01 | 2.04 | 1.87 | 2.04 |

For human hepatocyte incubation, the final concentration of DMSO was kept at 0.1%. Stock solution of isoflavone mix was diluted with hepatocyte incubation media from XenoTech at 2× concentration and then incubated in the CO2 incubator for 30 min before usage. Frozen female human hepatocytes were thawed and isolated followed XenoTech protocols. Hepatocytes were diluted with hepatocyte incubation media at final density of 2E6 cells/ml and incubated in CO₂ incubator for 30 min before usage. To start the reaction, equal volume of per-incubated isoflavone solution was added to hepatocyte solution. Incubation time was decided through preliminary trials to aim at the disappearance of 50% of isoflavones. To stop the reaction, equal volume of 50% acetonitrile and 50% methanol was added to the reaction mix. Samples were analyzed with LC/MS.

FIG. 6 shows that metabolism of the mixtures (Table 3) by human intestinal microsomes: biochanin A (5.41E-4 ml/min/mg protein)>genistein (4.28E-4 ml/min/mg protein)>equol (1.07E-4 ml/min/mg protein)>formononetin (7.31E-5 ml/min/mg protein)>daidzein (6.32E-5 ml/min/mg protein).

FIG. 7 shows the rate of metabolism of the mixtures by human hepatocytes. The rates are: equol (1.21E-5 ml/min/million cells)>biochanin A (8.88E-6 ml/min/million cells)>genistein (5.14E-6 ml/min/million cells)>daidzein (4.07E-6 ml/min/million cells)>formononetin (3.45E-6 ml/min/million cells).

From these studies, it is clearly shown that there are no metabolic interactions between the five components. In these metabolic studies, no Phase I metabolites were detected suggesting that the formation of Phase I metabolites, such as daidzein and genistein are formed in the intestinal lumen. This piece of information is important in that the rate of formation of these metabolites is dependent on the solubility of formononetin and biochanin A. These results are consistent with that reported by Howes et al. (2002) in that the peak time of the Phase I metabolites is delayed.

### Example 5

The objective of this study is to examine the rate of conversion of glucuronide conjugates of the phytoestrogen back into their respectively aglycone in the gut.

The plasma profiles of the conjugated phytoestrogens after the administration of Red clover show the characteristics of a “hump”, which usually suggest enterohepatic recycling. This hypothesis was confirmed when Schneider (2000) showed that glucuronide conjugates can be converted back to its aglycone before it is absorbed again. In order to capture this recycling process, quantitative data is needed to show the importance of this pathway. Schneider's method will be modified for this study.

The enterohepatic cycling process was simulated in silico and is incorporated to the PBPKPD model.

### Example 6

The protocol used by Moon et al. (2006) will be used for measuring plasma protein binding of individuals. Parameters will be used for PPT simulation. It is expected that the predominant components in the plasma are conjugates of biochanin A, formononetin, genistein, daidzein and equol. The respective aglycone will constitute less than 5% of the total concentration. Plasma protein binding of the aglycones was found to be approximately 95% for formononetin, biochanin A, daidzein, genistein and equol.

### Example 7

One objective of this example is to evaluate the components in the biophase such as mammary glands, uterus, heart and bone. Both in vitro and in vivo studies will be performed. The object of the in vivo study is to measure components that may be active in the biophase. Active components are identified from the results of Examples 5 and 6. This study is considered to provide a dramatic improvement on the PBPKPD models. The reasons are: 1. The actual components in biophase are revealed; 2. The number of components in the biophase is expected to be a tiny fraction of that generated in the fecal, intestinal and hepatic metabolism studies; 3. This will dramatically improve the certainty of statistical and in silico estimation of the time course of active components in the biophase.

Heart, kidneys, uterus/prostate, bone tissues will be harvested from a male/female rat after sacrifice. These tissues will be incubated with relevant mixtures of the bioactives. The rationale for employing the in vitro distribution study is to evaluate potential metabolism at the biophase and to estimate the free fraction of bioactives in the biophase. The results of this study will provide potential interactions between the bioactives and relevant distribution parameters to the biophase. These parameters are essential for constructing a meaningful biophase in the in silico model of PPT.

According to the literature, the half-life of genistein in bone is a lot higher than that in plasma. It is quite possible that the plasma half-life is not indicative of that of the time course in the biophase. The long half-life observed in the bone also suggests that, significant accumulation will occur with chronic dosing. The actual steady-state of bone concentration may not be reached until a couple of weeks after the initiation of dosing.

### Example 8

The results from examples 1 to 4 clearly showed that the active moieties of red clover are potentially formononetin, biochanin A, daidzein, genistein and equol. The objective of this example is to search for an optimal mixture of these five components that maximizes bone formation and minimizes bone resorption.

Homeostasis of the bone is governed by dynamic processes that involve osteoblasts and osteoclasts. Endogenous agents, like calcium ions (Ca²⁺), parathyroid hormone (PTH), calcitriol, glucocorticoids, estrogen, cytokines such as interleukin-6 (IL-6), tumor necrosis factor (TNF)-alpha and -beta, fibroblast growth factor (FGF), and calcitonin, are known to regulate the balance between osteoblasts and osteoclasts. Estrogen is known to enhance the growth and differentiation of osteoblasts (bone growth), while parathyroid hormone to the growth and differentiation of osteoclasts (bone resorption). Osteoblasts stimulated by estrogen can produce osteoproteoglygan (OPG) to regulate the differentiation of osteoclasts (Eriksen 2010).

Isoflavones are plant materials with estrogen-like structure and activity. Numerous animal/human studies using plant extracts, particularly red clover extracts, showed that isoflavones have beneficial effects on the condition of osteoporosis (Setchell and Lydeking-Olsen 2003; Nelson, Vesco et al. 2006). In order to understand the effect of red clover extract on bone growth, individual isoflavones were purified and test for their effects in in vitro studies. Isoflavones like formononetin, biochanin A, daidzein, and genistein are able to enhance differentiation and mineralization of osteoblast cell lines in vitro (Sugimoto and Yamaguchi 2000; Sugimoto and Yamaguchi 2000; Chen, Garner et al. 2003; Wende, Krenn et al. 2004; Lee and Choi 2005; Dong, Zhao et al. 2006; Ji, Zhao et al. 2006). The effective concentration varied among studies, even for the same isoflavones. The major drawback of these published results is that the culture media used contained estrogen or substances with estrogen activity. There are two sources of estrogen activity in the media: the first one is from fetal bovine serum and the second one is from phenol red (Berthois, Katzenellenbogen et al. 1986).

Isoflavones have also been shown to inhibit the differentiation osteoclasts and/or the bone resorption (Garcia Palacios, Robinson et al. 2005). Taken together, the effects of isoflavones on bone growth are achieved by promoting the differentiation/activity of osteoblasts and inhibiting the differentiation/activity of osteoclasts.

In the current example, we demonstrate the effects of isoflavones on osteoblasts and osteoclasts using tissue culture media containing serum treated with charcoal-dextran and phenol red free MEM. Presumably, this media has minimal contamination by substances carrying estrogenic activities.

### Materials and Methods

### Cell Culture:

MC3T3-E1 and Raw264.7 cells were purchased from American Type Culture Collection (ATCC). Both were maintained in MEM media supplemented with 5% fetal bovine serum, 2 mM L-glutamine, and antibiotics. All tissue culture media were purchased from Invitrogen/GIBCO. Isoflavones (Biochanin A, Daidzein, Equol, Formononetin, and Genistein) were purchased from Chromadex. Charcoal-dextran treated fetal bovine serum, RANKL and MCSF were purchased from Sigma-Aldrich. All other chemicals were purchased from Sigma-Aldrich unless indicated otherwise.

### Osteoblast Studies:

MC3T3-E1 cells were seeded at 5E4 cells/well in 48-well plates 24 hours prior to the addition of testing media. The testing media contain phenol red free MEM, 5% charcoal-dextran treated fetal bovine serum, 2 mM of L-glutamine, 5 mM glycerol-phosphate, 50 μg/ml of vitamin C and 1 μM of isoflavones containing biochanin A, daidzein, equol, formononetin, and genistein.

Media were changed twice a week for a total of 4 weeks. At the end of study, cell numbers were measured with a plate reader at 405 nm (reference wavelength 620 nm) after a 30-min incubation of XTT. Alkaline phosphatase activity was measured with a plate reader at 405 nm after the incubation of p-nitrophenyl phosphate in 0.1 M glycine buffer at pH 10.5.

### Osteoclast Studies:

Raw 264.7 cells were seeded at 1E3 cells/well in 96-well plates 24 hours prior to the addition of testing media. The testing media contain phenol red free MEM, 5% charcoal-dextran treated fetal bovine serum, 2 mM of L-glutamine, 50 ng/ml of RANKL, 5 ng/ml of MCSF, and 1 μM of isoflavones. Culture media were changed very other day for 5 days. At the end of study, cell numbers were measured with a plate reader at 405 nm (reference wavelength 620 nm) after a 1-hour incubation of XTT. Tartrate resistant acid phosphatase (TRAP) activity was measured with a plate reader at 405 nm after the incubation of p-nitrophenyl phosphate in a Sigma Acid Phosphatase, Leukocyte (TRAP) Kit.

A total of 121 combinations of the five isoflavones were created to cover all possible permutations. The incremental change was 20% and the maximal change of each isoflavone was 80% (Table 4 and FIG. 8). The dose ratio distribution for each isoflavone was described by a power law distribution (FIG. 8).

TABLE 4
Ratio of isoflavone (Biochanin A, Daidzein, Equol, Formononetin,
and Genistein) in the mixture combination. Total concentration
of isoflavones is 1 μM. The numbers under a component are
the fraction of composition of each component.

| | Isoflavones | | | | |
|---|---|---|---|---|---|
| Mixture | Biochanin | | | | |
| combination | A | Daidzein | Equol | Formononetin | Genistein |
| RC01 | 0 | 0 | 0.6 | 0 | 0.4 |
| RC02 | 0 | 0.2 | 0.2 | 0.6 | 0 |
| RC03 | 0 | 0 | 0.2 | 0.8 | 0 |
| RC04 | 0 | 0.4 | 0 | 0.4 | 0.2 |
| RC05 | 0 | 0.4 | 0.6 | 0 | 0 |
| RC06 | 0 | 0.2 | 0.4 | 0.4 | 0 |
| RC07 | 0 | 0.2 | 0.6 | 0.2 | 0 |
| RC08 | 0 | 0.2 | 0.4 | 0 | 0.4 |
| RC09 | 0 | 0.2 | 0 | 0.2 | 0.6 |
| RC10 | 0 | 0.4 | 0.2 | 0 | 0.4 |
| RC11 | 0 | 0 | 0.6 | 0.2 | 0.2 |
| RC12 | 0 | 0.6 | 0.2 | 0.2 | 0 |
| RC13 | 0 | 0.6 | 0 | 0 | 0.4 |
| RC14 | 0 | 0.4 | 0 | 0 | 0.6 |
| RC15 | 0 | 0.2 | 0.8 | 0 | 0 |
| RC16 | 0 | 0.4 | 0.2 | 0.4 | 0 |
| RC17 | 0 | 0 | 0.2 | 0.4 | 0.4 |
| RC18 | 0 | 0.8 | 0 | 0.2 | 0 |
| RC19 | 0 | 0 | 0.4 | 0 | 0.6 |
| RC20 | 0 | 0 | 0.4 | 0.4 | 0.2 |
| RC21 | 0 | 0.4 | 0.4 | 0 | 0.2 |
| RC22 | 0 | 0.4 | 0.4 | 0.2 | 0 |
| RC23 | 0 | 0.2 | 0.2 | 0 | 0.6 |
| RC24 | 0 | 0 | 0.8 | 0 | 0.2 |
| RC25 | 0 | 0.2 | 0.4 | 0.2 | 0.2 |
| RC26 | 0 | 0.2 | 0.2 | 0.2 | 0.4 |
| RC27 | 0 | 0.2 | 0 | 0.6 | 0.2 |
| RC28 | 0 | 0.2 | 0 | 0.4 | 0.4 |
| RC29 | 0 | 0 | 0 | 0.6 | 0.4 |
| RC30 | 0 | 0.6 | 0.4 | 0 | 0 |
| RC31 | 0 | 0 | 0 | 0.2 | 0.8 |
| RC32 | 0 | 0.4 | 0 | 0.2 | 0.4 |
| RC33 | 0 | 0.8 | 0.2 | 0 | 0 |
| RC34 | 0 | 0.2 | 0 | 0.8 | 0 |
| RC35 | 0 | 0 | 0 | 0.8 | 0.2 |
| RC36 | 0 | 0.6 | 0.2 | 0 | 0.2 |
| RC37 | 0 | 0 | 0.2 | 0.2 | 0.6 |
| RC38 | 0 | 0 | 0.4 | 0.6 | 0 |
| RC39 | 0 | 0 | 0.2 | 0.6 | 0.2 |
| RC40 | 0 | 0 | 0.8 | 0.2 | 0 |
| RC41 | 0 | 0.2 | 0.2 | 0.4 | 0.2 |
| RC42 | 0 | 0.2 | 0.6 | 0 | 0.2 |
| RC43 | 0 | 0 | 0.2 | 0 | 0.8 |
| RC44 | 0 | 0.6 | 0 | 0.4 | 0 |
| RC45 | 0 | 0.2 | 0 | 0 | 0.8 |
| RC46 | 0 | 0 | 0 | 0.4 | 0.6 |
| RC47 | 0 | 0.8 | 0 | 0 | 0.2 |
| RC48 | 0 | 0.6 | 0 | 0.2 | 0.2 |
| RC49 | 0 | 0.4 | 0.2 | 0.2 | 0.2 |
| RC50 | 0 | 0.4 | 0 | 0.6 | 0 |
| RC51 | 0 | 0 | 0.4 | 0.2 | 0.4 |
| RC52 | 0 | 0 | 0.6 | 0.4 | 0 |
| RC53 | 0.2 | 0.4 | 0.2 | 0.2 | 0 |
| RC54 | 0.2 | 0 | 0.6 | 0.2 | 0 |
| RC55 | 0.2 | 0.6 | 0 | 0.2 | 0 |
| RC56 | 0.2 | 0 | 0.2 | 0.2 | 0.4 |
| RC57 | 0.2 | 0 | 0 | 0.8 | 0 |
| RC58 | 0.2 | 0.2 | 0.4 | 0.2 | 0 |
| RC59 | 0.2 | 0.2 | 0 | 0.2 | 0.4 |
| RC60 | 0.2 | 0.4 | 0.2 | 0 | 0.2 |
| RC61 | 0.2 | 0 | 0.6 | 0 | 0.2 |
| RC62 | 0.2 | 0 | 0.2 | 0 | 0.6 |
| RC63 | 0.2 | 0.2 | 0.4 | 0 | 0.2 |
| RC64 | 0.2 | 0.4 | 0 | 0 | 0.4 |
| RC65 | 0.2 | 0.6 | 0.2 | 0 | 0 |
| RC66 | 0.2 | 0.2 | 0 | 0.4 | 0.2 |
| RC67 | 0.2 | 0 | 0 | 0.2 | 0.6 |
| RC68 | 0.2 | 0.2 | 0.2 | 0.2 | 0.2 |
| RC69 | 0.2 | 0 | 0.4 | 0 | 0.4 |
| RC70 | 0.2 | 0.4 | 0 | 0.2 | 0.2 |
| RC71 | 0.2 | 0 | 0 | 0 | 0.8 |
| RC72 | 0.2 | 0.6 | 0 | 0 | 0.2 |
| RC73 | 0.2 | 0.8 | 0 | 0 | 0 |
| RC74 | 0.2 | 0.4 | 0 | 0.4 | 0 |
| RC75 | 0.2 | 0 | 0.4 | 0.4 | 0 |
| RC76 | 0.2 | 0 | 0.8 | 0 | 0 |
| RC77 | 0.2 | 0.2 | 0 | 0 | 0.6 |
| RC78 | 0.2 | 0 | 0.4 | 0.2 | 0.2 |
| RC79 | 0.2 | 0 | 0.2 | 0.4 | 0.2 |
| RC80 | 0.2 | 0 | 0 | 0.4 | 0.4 |
| RC81 | 0.2 | 0.4 | 0.4 | 0 | 0 |
| RC82 | 0.2 | 0.2 | 0 | 0.6 | 0 |
| RC83 | 0.2 | 0.2 | 0.2 | 0.4 | 0 |
| RC84 | 0.2 | 0.2 | 0.2 | 0 | 0.4 |
| RC85 | 0.2 | 0.2 | 0.6 | 0 | 0 |
| RC86 | 0.2 | 0 | 0 | 0.6 | 0.2 |
| RC87 | 0.2 | 0 | 0.2 | 0.6 | 0 |
| RC88 | 0.4 | 0.4 | 0.2 | 0 | 0 |
| RC89 | 0.4 | 0.2 | 0 | 0 | 0.4 |
| RC90 | 0.4 | 0.2 | 0.4 | 0 | 0 |
| RC91 | 0.4 | 0 | 0.6 | 0 | 0 |
| RC92 | 0.4 | 0.2 | 0 | 0.4 | 0 |
| RC93 | 0.4 | 0 | 0 | 0.6 | 0 |
| RC94 | 0.4 | 0.4 | 0 | 0 | 0.2 |
| RC95 | 0.4 | 0.6 | 0 | 0 | 0 |
| RC96 | 0.4 | 0.2 | 0.2 | 0.2 | 0 |
| RC97 | 0.4 | 0 | 0.2 | 0.4 | 0 |
| RC98 | 0.4 | 0 | 0 | 0 | 0.6 |
| RC99 | 0.4 | 0.2 | 0.2 | 0 | 0.2 |
| RC100 | 0.4 | 0 | 0.2 | 0 | 0.4 |
| RC101 | 0.4 | 0 | 0.4 | 0.2 | 0 |
| RC102 | 0.4 | 0 | 0.4 | 0 | 0.2 |
| RC103 | 0.4 | 0.4 | 0 | 0.2 | 0 |
| RC104 | 0.4 | 0 | 0 | 0.2 | 0.4 |
| RC105 | 0.4 | 0 | 0.2 | 0.2 | 0.2 |
| RC106 | 0.4 | 0.2 | 0 | 0.2 | 0.2 |
| RC107 | 0.4 | 0 | 0 | 0.4 | 0.2 |
| RC108 | 0.6 | 0.4 | 0 | 0 | 0 |
| RC109 | 0.6 | 0 | 0.2 | 0 | 0.2 |
| RC110 | 0.6 | 0.2 | 0 | 0 | 0.2 |
| RC111 | 0.6 | 0 | 0 | 0.4 | 0 |
| RC112 | 0.6 | 0 | 0.4 | 0 | 0 |
| RC113 | 0.6 | 0 | 0.2 | 0.2 | 0 |
| RC114 | 0.6 | 0 | 0 | 0.2 | 0.2 |
| RC115 | 0.6 | 0.2 | 0.2 | 0 | 0 |
| RC116 | 0.6 | 0.2 | 0 | 0.2 | 0 |
| RC117 | 0.6 | 0 | 0 | 0 | 0.4 |
| RC118 | 0.8 | 0.2 | 0 | 0 | 0 |
| RC119 | 0.8 | 0 | 0 | 0.2 | 0 |
| RC120 | 0.8 | 0 | 0.2 | 0 | 0 |
| RC121 | 0.8 | 0 | 0 | 0 | 0.2 |

Isoflavones were first dissolved in DMSO and stock solutions of the mixtures were prepared as listed on Table 4. The total concentration of each stock solution was 1 mM.

### Method of Analysis:

Osteoblast and osteoclast differentiations were quantified. ALP is highly expressed by the mature osteoblasts and ACP is expressed by osteoclasts. Values of XTT serve as a correction for the difference in cell numbers. Therefore, ALP/XTT and ACP/XTT ratios are used to quantify osteoblast and osteoclast activities. The upper panel of FIG. 9 shows a plot of the normalized ACP vs. ALP of the 121 combinations. The lower panel is the activity of pure isoflavone measured at 1 μM.

Since the aim of this study is to identify the optimal mixture(s), which has the highest activity in, enhancing bone formation and inhibiting bone resorption. The strategy is divide the data presented in FIG. 9 into four quadrants: Q1, Q2, Q3 and Q4. The division is achieved using the average values of ACP and ACP ratios (dotted lines of FIG. 9). Q₁ has data of 29 mixtures. These mixtures have the highest ALP and minimum ACP values. The optimal mixture(s) is present in this quadrant. Q₄ has data of 23 mixtures. These mixtures have the least favorable for bone formation and resorption. Q₂ has 37 mixtures. These mixtures, like that of Q₁, have the highest bone forming activity, but they also least activity against bone resorption. Q₃ has 32 mixtures. These mixtures have the lowest activity for bone formation but most effective against bone resorption.

Since the dose ratio distribution histogram for all mixtures is shown in FIG. 8 and the distribution follows a power law distribution. This distribution suggests that all possible interactions have equal probability. A similar approach is used to qualitatively identify the behavior of pure isoflavones and their mixtures in different quadrants.

Difference in relative entropy of two distributions, P_{Qi}(k) and P_{Qj}(k), defined as S(P_{Qi},P_{Qj})=−τₖP_{Qi}(k)log P_{Qi}(k)/P_{Qj}(k) is used to quantify information differences between distributions (Tseng 2006). The higher the relative entropy the higher is the similarity between the two distributions: P_{Qi}(k) and P_{Qj}(k). This approach is employed to evaluate relative entropy of normalized histograms of each component in paired quadrants which share common activity; for example, Q₁ and Q₂ are osteoblast producers (FIG. 10). The relative entropy values for each isoflavone are listed on Table 5. A comparison of entropy values between opposing effects, for example, high and low ALP producers, reveal the effect of each component. Note, the non-diagonal terms such as (Q₁,Q₄) and (Q₂,Q₃) pairs are excluded because those terms may involve more complicated ACP and ALP interactions. Based on this quantity and property of the four quadrants, the effects of each isoflavone are statistically compared.

### Results and Discussions:

Using PPT, five isoflavones: biochanin A, formononetin, genistein, daidzein and equol have been identified as active moieties. The ultimate goal of this study is to search for optimal combination(s), which is most effective in preventing/treating osteoporosis. All possible combinations were studied in order to cover potential interactions induced by each isoflavone (Table 4 and FIG. 8).

The optimal candidates should possess the highest osteoblast production (highest ALP/XTT ratios) and the lowest osteoclast activity (lowest ACP/XTT ratios). FIG. 9 is constructed to facilitate the identification of the optimal candidates. The figure is divided into four quadrants. Candidates in Q₁ have the highest ALP and lowest ACP ratios, suggesting that the optimal candidates are present in this quadrant. The most ineffective candidates are found in Q₄ where candidates have the lowest osteoblast activity and highest osteoclast counts. Q₂ candidates have the highest osteoblast and osteoclast activity, not ideal as optimal candidates. Q₃ contains candidates that are most effective for osteoclast inhibition, but these candidates have the lowest osteoblast forming ability.

Genistein was identified to be the most potent among the five isoflavones in bone formation (FIG. 9, lower panel). The activity for bone formation was not significantly different among the rest of the four isoflavones, although equol has a higher mean value. Data presented on FIG. 9 show that candidates in Q₁ have a higher potency than genistein alone, suggesting synergistic interactions.

A detailed analysis of the distribution of individual isoflavones in the combinations (FIG. 10) showed that majority of combinations in Q₁ do not contain formononetin and at least 50% of combinations in Q₂, Q₃, or Q₄ contain formononetin, indicating formononetin is not required.

Table 5 shows that biochanin A is required for osteoblast formation and osteoclast inhibition. This is due to its relatively high entropy values for (Q₁, Q₂) and (Q₁, Q₃) as opposed to (Q₃, Q₄) and (Q₂, Q₄), respectively. This observation is further supported by the fact that there are 65% of mixtures in Q₄ quadrant have no biochanin A; the quadrant contains the least effective group of mixtures. Mixtures in Q₁, Q₂ and Q₃ have either no or very few mixtures that have 80% biochanin A. This observation suggests that a lower dose of biochanin A is preferable for optimal effects.

TABLE 5
Relative entropy S(P_{Qi}, P_{Qj})

| Common property | High ALP | Low ALP | High ACP | Low ACP |
|---|---|---|---|---|
| (Quadrant pair) | (Q1, Q2) | (Q3, Q4) | (Q2, Q4) | (Q1, Q3) |
| Biochanin A | −0.18 | −28.32 | −74.52 | −0.36 |
| Daidzein | −0.25 | −14.39 | −37.35 | −0.12 |
| Equol | −0.03 | −28.65 | −12.41 | −0.06 |
| Formononetin | −31.71 | 0 | −0.25 | −0.15 |
| Genistein | −31.57 | −42.81 | −61.95 | −31.66 |

Similar to biochanin A, daidzein and equol shared similar trends of relative entropy, suggesting that these two isoflavones are required for the optimum mixture.

Formononetin has the highest entropy for low ALP production (Table 5), suggesting its presence in a mixture is not desirable. The effects of formononetin on osteoclast are likely to be ineffective because the relative entropy values between induction and inhibition of osteoclast inhibition are not distinct. It is safe to conclude that formononetin is not required in the mixture.

The role of genistein in osteoblast formation and osteoclast inhibition is mild (Table 5). More refined studies are required to clarify the role of genistein in the mixture. Since genistein is the most potent compound in its pure form, it is included in the mixture of combinations.

The above relative entropy analysis reveals the effects of each isoflavone under involvement of possible synergism and antagonism on ALP and ACP. We can summarize that the preferred combinations may primarily contain daidzein, equol and small amount of biochanin A. Genistein may be included. Yet the dose ratio may not be an important issue. Formononetin should not be included due to its ambiguous role in ACP and the tendency of decreasing ALP. As shown in our data, two combinations from the Q1 quadrant, 20% biochanin A, 20% daidzein and 60% equol and 40% daidzein, 40% equol and 20% genistein, do generate high osteoblast and low osteoclast (ALP/XTT=0.67±0.03, ACP/XTT=0.66±0.07) and (ALP/XTT=0.7±0.02, ACP/XTT=0.67±0.06) respectively.

### Example 9

The objective of this example is to use data generated in examples 1 to 8 to estimate the optimum dosage of biochanin A, daidzein, genistein and equol in the human body. The goal is produce a set of blood curves, which provides the ideal proportions of these components in the body.

### Example 10

The purpose of this example is to establish optimal ratios of the isoflavones so that a product can be designed to produce maximal effect for preventing/treating osteoporosis.

It is generally believed that red clover is better than soy for the prevention of osteoporosis. However, the research conducted using PPT suggests that red clover is not ideal for the prevention/treatment of osteoporosis either.

The pharmacokinetic study of PROMENSIL®, a red clover extract (Howes et al. 2002), shows the blood concentrations of biochanin A, daidzein, formononetin, and genistein has a ratio of 18:29:4:49 when calculated using AUC values. Although we did not have combinations with an exact ratio of these isoflavones in our test set, the closest one is 20:20:20:40. This ratio shows up in Q₄, a quadrant where least ideal combinations are found. The presence of fomononetin in the combination seems to be the culprit because of its negative role on ALP and its ambiguous role on ACP.

The study conducted by Mathey et al (2006) using a soy product shows that ratio of plasma concentration of daidzein:equol:genistein is 35:9:56. The closest combinations in our test set are (40:20:40) and (40:0:60). These two combinations are not found in Q₁, the quadrant where effective mixtures are found. Therefore, there is no surprise that clinical trial results on soy products are often equivocal.

Five most effective combinations found in Q₁ have ratios of biochanin A, daidzein, equol, formononetin, and genistein in the form of (0:20:80:0:0), (0:40:40:0:20), (20:0:0:0:80), (20:60:0:0:20), and (60:0:0:0:40). These isoflavone combinations do not exist in nature.

Based on the current results, the preferred combinations consist primarily of daidzein, equol, small amount of biochanin A and genistein. Dose ratio may not be an important issue, however, formononetin should be avoided.

Two combinations from Q₁, 20% biochanin A, 20% daidzein and 60% equol and 40% daidzein, 40% equol and 20% genistein, generate high osteoblast (ALP/XTT: 0.67±0.03 and 0.7±0.02) and low osteoclast (ACP/XTT: 0.66±0.07 and 0.67±0.06) values.

Despite the importance placed on formononetin in designing a red clover product, our results show that formononetin should be avoided, although the beneficial effects of formononetin are linked to its conversion into active metabolites such as daidzein and equol. However, the reliance on the body's ability to produce active metabolites by gut flora is not ideal because inter-individual variability is huge. This could be one of the major causes of inconsistencies of red clover products.

Based on these data, an ideal product of a phytoestrogen mix would require fractional purification of red clover to obtain biochanin A, and soy to obtain daidzein and genistein. Equol will have to be added since the human body does not produce equol in such a high proportion. Furthermore, half to two third of the human population do not produce equol from formononetin and daidzein.

### REFERENCES

Abrams, S. A., I. J. Griffin, et al. (2002). “Using stable isotopes to assess the bioavailability of minerals in food fortification programs.” Food Nutr Bull 23(3 Suppl): 158-165.
Allred, C. D., K. F. Allred, et al. (2004). “Dietary genistein results in larger MNU-induced, estrogen-dependent mammary tumors following ovariectomy of Sprague-Dawley rats.” Carcinogenesis 25(2): 211-218.
Atkinson, C., J. E. Compston, et al. (2004). “The effects of phytoestrogen isoflavones on bone density in women: a double-blind, randomized, placebo-controlled trial.” Am J Clin Nutr 79(2): 326-333.
Baillard, C., A. Bianchi, et al. (2007). “[Anaesthetic preoperative assessment of chronic medications and herbal medicine use: a multicenter survey].” Ann Fr Anesth Reanim 26(2): 132-135.
Beck, V., U. Rohr, et al. (2005). “Phytoestrogens derived from red clover: an alternative to estrogen replacement therapy?” J Steroid Biochem Mol Biol 94(5): 499-518.
Beck, V., E. Unterrieder, et al. (2003). “Comparison of hormonal activity (estrogen, androgen and progestin) of standardized plant extracts for large scale use in hormone replacement therapy.” J Steroid Biochem Mol Biol 84(2-3): 259-268.

Berthois, Y., J. A. Katzenellenbogen, et al. (1986). “Phenol red in tissue culture media is a weak estrogen: implications concerning the study of estrogen-responsive cells in culture.” Proc Natl Acad Sci USA 83(8): 2496-2500.
Booth, N. L., C. R. Overk, et al. (2006). “Seasonal variation of red clover (Trifolium pratense L., Fabaceae) isoflavones and estrogenic activity.” J Agric Food Chem 54(4): 1277-1282.
Booth, N. L., C. E. Piersen, et al. (2006). “Clinical studies of red clover (Trifolium pratense) dietary supplements in menopause: a literature review.” Menopause 13(2): 251-264.
Bowey, E., H. Adlercreutz, et al. (2003). “Metabolism of isoflavones and lignans by the gut microflora: a study in germ-free and human flora associated rats.” Food Chem Toxicol 41(5): 631-636.
Cassidy, A., P. Albertazzi, et al. (2006). “Critical review of health effects of soyabean phyto-oestrogens in post-menopausal women.” Proc Nutr Soc 65(1): 76-92.
Chang, H. C., M. I. Churchwell, et al. (2000). “Mass spectrometric determination of Genistein tissue distribution in diet-exposed Sprague-Dawley rats.” J Nutr 130(8): 1963-1970.
Chen, J., H. Lin, et al. (2003). “Metabolism of flavonoids via enteric recycling: role of intestinal disposition.” J Pharmacol Exp Ther 304(3): 1228-1235.
Chen, J., H. Lin, et al. (2005). “Absorption and metabolism of genistein and its five isoflavone analogs in the human intestinal Caco-2 model.” Cancer Chemother Pharmacol 55(2): 159-169.
Chen, J., S. Wang, et al. (2005). “Disposition of flavonoids via recycling: comparison of intestinal versus hepatic disposition.” Drug Metab Dispos 33(12): 1777-1784.
Chen, X., S. C. Garner, et al. (2003). “Effects of genistein on expression of bone markers during MC3T3-E1 osteoblastic cell differentiation.” J Nutr Biochem 14(6): 342-349.
Clifton-Bligh, P. B., R. J. Baber, et al. (2001). “The effect of isoflavones extracted from red clover (Rimostil) on lipid and bone metabolism.” Menopause 8(4): 259-265.
Coldham, N. G. and M. J. Sauer (2000). “Pharmacokinetics of [(14)C]Genistein in the rat: gender-related differences, potential mechanisms of biological action, and implications for human health.” Toxicol Appl Pharmacol 164(2): 206-215.
Day, A. J., M. S. DuPont, et al. (1998). “Deglycosylation of flavonoid and isoflavonoid glycosides by human small intestine and liver beta-glucosidase activity.” FEBS Lett 436(1): 71-75.
Dong, T. T., K. J. Zhao, et al. (2006). “Chemical and biological assessment of a chinese herbal decoction containing Radix Astragali and Radix Angelicae Sinensis: Determination of drug ratio in having optimized properties.” J Agric Food Chem 54(7): 2767-2774.
Engelhardt, P. F. and C. R. Riedl (2008). “Effects of one-year treatment with isoflavone extract from red clover on prostate, liver function, sexual function, and quality of life in men with elevated PSA levels and negative prostate biopsy findings.” Urology 71(2): 185-190; discussion 190.
Engelhardt, P. F. and C. R. Riedl (2008). “Reply to editorial comment, Re: Engelhardt P F and Riedl C R, Effects of one-year treatment with isoflavone extract from red clover on prostate, liver function, sexual function, and quality of life in men with elevated PSA levels and negative prostate biopsy findings. Urology 71: 190, 2008.” Urology 71(5): 987.
Eriksen, E. F. (2010). “Cellular mechanisms of bone remodeling.” Rev Endocr Metab Disord 11(4): 219-227.
Fernandez, E., S. Gallus, et al. (2003). “Hormone replacement therapy and cancer risk: a systematic analysis from a network of case-control studies.” Int J Cancer 105(3): 408-412.
Former, P., C. Theurer, et al. (2006). “Visualization and analysis of the release mechanism of shellac coated ascorbic acid pellets.” Pharmazie 61(12): 1005-1008.
Fujita, T. and M. Fukase (1992). “Comparison of osteoporosis and calcium intake between Japan and the United States.” Proc Soc Exp Biol Med 200(2): 149-152.
Gambacciani, M., M. Ciaponi, et al. (2007). “The HRT misuse and osteoporosis epidemic: a possible future scenario.”Climacteric 10(4): 273-275.
Gambacciani, M., P. Monteleone, et al. (2003). “Hormone replacement therapy and endometrial, ovarian and colorectal cancer.” Best Pract Res Clin Endocrinol Metab 17(1): 139-147.
Garcia Palacios, V., L. J. Robinson, et al. (2005). “Negative regulation of RANKL-induced osteoclastic differentiation in RAW264.7 Cells by estrogen and phytoestrogens.” J Biol Chem 280(14): 13720-13727.
Ge, Y., D. Chen, et al. (2006). “Enhancing effect of daidzein on the differentiation and mineralization in mouse osteoblast-like MC3T3-E1 cells.” Yakugaku Zasshi 126(8): 651-656.
Gu, L., M. Laly, et al. (2005). “Isoflavone conjugates are underestimated in tissues using enzymatic hydrolysis.” J Agric Food Chem 53(17): 6858-6863.
Hale, G. E., C. L. Hughes, et al. (2001). “A double-blind randomized study on the effects of red clover isoflavones on the endometrium.” Menopause 8(5): 338-346.
Heinonen, S. M., K. Wahala, et al. (2004). “Identification of urinary metabolites of the red clover isoflavones formononetin and biochanin A in human subjects.” J Agric Food Chem 52(22): 6802-6809.
Howes, J., M. Waring, et al. (2002). “Long-term pharmacokinetics of an extract of isoflavones from red clover (Trifolium pratense).” J Altern Complement Med 8(2): 135-142.
Hur, H. and F. Rafii (2000). “Biotransformation of the isoflavonoids biochanin A, formononetin, and glycitein by Eubacterium limosum.” FEMS Microbiol Lett 192(1): 21-25.
Izumi, T., M. K. Piskula, et al. (2000). “Soy isoflavone aglycones are absorbed faster and in higher amounts than their glucosides in humans.” J Nutr 130(7): 1695-1699.
Ji, Z. N., W. Y. Zhao, et al. (2006). “In vitro estrogenic activity of formononetin by two bioassay systems.” Gynecol Endocrinol 22(10): 578-584.
Jia, X., J. Chen, et al. (2004). “Disposition of flavonoids via enteric recycling: enzyme-transporter coupling affects metabolism of biochanin A and formononetin and excretion of their phase II conjugates.” J Pharmacol Exp Ther 310(3): 1103-1113.
Kano, M., T. Takayanagi, et al. (2006). “Bioavailability of isoflavones after ingestion of soy beverages in healthy adults.” J Nutr 136(9): 2291-2296.
Kelly, G. E. (1998). Health supplements containing phto-estrogens, analogues or metabolites thereof. USPTO. USPTO. U.S.A., Novogen Research Pty. Ltd.
Kelly, G. E. (2000). Cardiovascular and bone treatment using isoflavones. World Intellectual Property Organization. W. I. P. Organization.
Kelly, G. E. (2002). Treatment or prevention of osteoporosis. USPTO. USPTO. U.S.A., Novogen, Inc. U.S. Pat. No. 6,340,703.
Kelly, G. E. (2003). Methods for treating or reducing predisposition to breast cancer, pre-menstrual syndrome or symptoms associated with menopause by administration of phyto-estrogen. USPTO. USPTO. U.S.A., Novogen Research Pty Limited.
Kuiper, G. G., B. Carlsson, et al. (1997). “Comparison of the ligand binding specificity and transcript tissue distribution of estrogen receptors alpha and beta.” Endocrinology 138(3): 863-870.
Lee, K. H. and E. M. Choi (2005). “Biochanin A Stimulates Osteoblastic Differentiation and Inhibits Hydrogen Peroxide-Induced Production of Inflammatory Mediators in MC3T3-E1 Cells.” Biol. Pharm. Bull. 28(10): 1948-1953.
Li, X. H., J. C. Zhang, et al. (2005). “Effect of daidzin, genistin, and glycitin on osteogenic and adipogenic differentiation of bone marrow stromal cells and adipocytic transdifferentiation of osteoblasts.” Acta Pharmacol Sin 26(9): 1081-1086.
Liu, J., J. E. Burdette, et al. (2001). “Evaluation of estrogenic activity of plant extracts for the potential treatment of menopausal symptoms.” J Agric Food Chem 49(5): 2472-2479.
Liu, Y. and M. Hu (2002). “Absorption and metabolism of flavonoids in the caco-2 cell culture model and a perused rat intestinal model.” Drug Metab Dispos 30(4): 370-377.
Ma, D. F., L. Q. Qin, et al. (2008). “Soy isoflavone intake inhibits bone resorption and stimulates bone formation in menopausal women: meta-analysis of randomized controlled trials.” Eur J Clin Nutr 62(2): 155-161.
Migliaccio, S. and J. J. Anderson (2003). “Isoflavones and skeletal health: are these molecules ready for clinical application?” Osteoporosis international: a journal established as result of cooperation between the European Foundation for Osteoporosis and the National Osteoporosis Foundation of the USA 14(5): 361-368.
Moon, Y. J., K. Sagawa, et al. (2006). “Pharmacokinetics and bioavailability of the isoflavone biochanin A in rats.” Aaps J 8(3): E433-442.
Nagel, S. C., F. S. vom Saal, et al. (1998). “The effective free fraction of estradiol and xenoestrogens in human serum measured by whole cell uptake assays: physiology of delivery modifies estrogenic activity.” Proc Soc Exp Biol Med 217(3): 300-309.
Nelson, H. D., K. K. Vesco, et al. (2006). “Nonhormonal therapies for menopausal hot flashes: systematic review and meta-analysis.” Jama 295(17): 2057-2071.
Overk, C. R., P. Yao, et al. (2005). “Comparison of the in vitro estrogenic activities of compounds from hops (Humulus lupulus) and red clover (Trifolium pratense).” J Agric Food Chem 53(16): 6246-6253.
Pearnchob, N. and R. Bodmeier (2003). “Dry polymer powder coating and comparison with conventional liquid-based coatings for Eudragit) RS, ethylcellulose and shellac.” Eur J Pharm Biopharm 56(3): 363-369.
Pearnchob, N., A. Dashevsky, et al. (2004). “Improvement in the disintegration of shellac-coated soft gelatin capsules in simulated intestinal fluid.” J Control Release 94(2-3): 313-321.
Pearnchob, N., J. Siepmann, et al. (2003). “Pharmaceutical applications of shellac: moisture-protective and taste-masking coatings and extended-release matrix tablets.” Drug Dev Ind Pharm 29(8): 925-938.
Pike, A. C., A. M. Brzozowski, et al. (1999). “Structure of the ligand-binding domain of oestrogen receptor beta in the presence of a partial agonist and a full antagonist.” Embo J 18(17): 4608-4618.
Rachon, D., A. Menche, et al. (2008). “Effects of dietary equol administration on the mammary gland in ovariectomized Sprague-Dawley rats.” Menopause 15(2): 340-345.
Rachon, D., T. Vortherms, et al. (2007). “Uterotropic effects of dietary equol administration in ovariectomized Sprague-Dawley rats.” Climacteric 10(5): 416-426.
Richelle, M., S. Pridmore-Merten, et al. (2002). “Hydrolysis of isoflavone glycosides to aglycones by beta-glycosidase does not alter plasma and urine isoflavone pharmacokinetics in postmenopausal women.” J Nutr 132(9): 2587-2592.
Rimoldi, G., J. Christoffel, et al. (2007). “Effects of chronic genistein treatment in mammary gland, uterus, and vagina.” Environ Health Perspect 115 Suppl 1: 62-68.
Rufer, C. E., R. Maul, et al. (2007). “In vitro and in vivo metabolism of the soy isoflavone glycitein.” Mol Nutr Food Res 51(7): 813-823.
Schneider, H., R. Simmering, et al. (2000). “Degradation of quercetin-3-glucoside in gnotobiotic rats associated with human intestinal bacteria.” J Appl Microbiol 89(6): 1027-1037.
Schult, T. M., K. E. Ensrud, et al. (2004). “Effect of isoflavones on lipids and bone turnover markers in menopausal women.” Maturitas 48(3): 209-218.
Seelig, M. S., B. M. Altura, et al. (2004). “Benefits and risks of sex hormone replacement in postmenopausal women.” J Am Coll Nutr 23(5): 482S-496S.
Sepehr, E., G. Cooke, et al. (2007). “Bioavailability of soy isoflavones in rats Part I: application of accurate methodology for studying the effects of gender and source of isoflavones.” Mol Nutr Food Res 51(7): 799-812.
Setchell, K. D., N. M. Brown, et al. (2001). “Bioavailability of pure isoflavones in healthy humans and analysis of commercial soy isoflavone supplements.” J Nutr 131(4 Suppl): 1362S-1375S.
Setchell, K. D., N. M. Brown, et al. (2003). “Bioavailability, disposition, and dose-response effects of soy isoflavones when consumed by healthy women at physiologically typical dietary intakes.” J Nutr 133(4): 1027-1035.
Setchell, K. D., N. M. Brown, et al. (2002). “Evidence for lack of absorption of soy isoflavone glycosides in humans, supporting the crucial role of intestinal metabolism for bioavailability.” Am J Clin Nutr 76(2): 447-453.
Setchell, K. D. and A. Cassidy (1999). “Dietary isoflavones: biological effects and relevance to human health.” J Nutr 129(3): 758S-767S.
Setchell, K. D., C. Clerici, et al. (2005). “S-equol, a potent ligand for estrogen receptor beta, is the exclusive enantiomeric form of the soy isoflavone metabolite produced by human intestinal bacterial flora.” Am J Clin Nutr 81(5): 1072-1079.
Setchell, K. D. and S. J. Cole (2006). “Method of defining equol-producer status and its frequency among vegetarians.” J Nutr 136(8): 2188-2193.
Setchell, K. D., M. S. Faughnan, et al. (2003). “Comparing the pharmacokinetics of daidzein and genistein with the use of 13C-labeled tracers in premenopausal women.” Am J Clin Nutr 77(2): 411-419.
Setchell, K. D. and E. Lydeking-Olsen (2003). “Dietary phytoestrogens and their effect on bone: evidence from in vitro and in vivo, human observational, and dietary intervention studies.” Am J Clin Nutr 78(suppl): 593S-609S.
Sugimoto, E. and M. Yamaguchi (2000). “Anabolic effect of genistein in osteoblastic MC3T3-E1 cells.” Int J Mol Med 5(5): 515-520.
Sugimoto, E. and M. Yamaguchi (2000). “Stimulatory effect of Daidzein in osteoblastic MC3T3-E1 cells.” Biochem Pharmacol 59(5): 471-475.
Tam, Y. K., J. Y.-C. Lin, et al. (2011). Preparation of botanical extracts containing absorbable components using pharmaceutical platform technology. USPTO. U.S.A.
Tam, Y. K. and J. A. Tuszynski (2008). Pharmaceutical platform technology for the development of natural products P. C. Treaty.
Thompson, L. U., B. A. Boucher, et al. (2007). “Dietary phytoestrogens, including isoflavones, lignans, and coumestrol, in nonvitamin, nonmineral supplements commonly consumed by women in Canada.” Nutr Cancer 59(2): 176-184.
To be, H., O. Komiyama, et al. (1997). “Daidzein stimulation of bone resorption in pit formation assay.” Biosci Biotechnol Biochem 61(2): 370-371.
Tolleson, W. H., D. R. Doerge, et al. (2002). “Metabolism of biochanin A and formononetin by human liver microsomes in vitro.” J Agric Food Chem 50(17): 4783-4790.
Tseng, C.-Y. (2006). “Entropic criterion for model selection.” Physica A 370(2): 530-538.
Tsunoda, N., S. Pomeroy, et al. (2002). “Absorption in humans of isoflavones from soy and red clover is similar.” J Nutr 132(8): 2199-2201.
Wang, S. W., J. Chen, et al. (2006). “Disposition of flavonoids via enteric recycling: structural effects and lack of correlations between in vitro and in situ metabolic properties.” Drug Metab Dispos 34(11): 1837-1848.
Wang, S. W., Y. Chen, et al. (2008). “Variable isoflavone content of red clover products affects intestinal disposition of biochanin a, formononetin, genistein, and daidzein.” J Altern Complement Med 14(3): 287-297.
Wende, K., L. Krenn, et al. (2004). “Red clover extracts stimulate differentiation of human osteoblastic osteosarcoma HOS58 cells.” Planta Med 70(10): 1003-1005.
Wuttke, W., H. Jarry, et al. (2007). “Isoflavones—safe food additives or dangerous drugs?” Ageing Res Rev 6(2): 150-188.
Yoshida, K., T. Tsukamoto, et al. (1985). “Disposition of ipriflavone (TC-80) in rats and dogs.” Radioisotopes 34(11): 618-623.
Zubik, L. and M. Meydani (2003). “Bioavailability of soybean isoflavones from aglycone and glucoside forms in American women.” Am J Clin Nutr 77(6): 1459-1465.

What is claimed is:

1. A method of identifying compositions for treating or preventing osteoporosis comprising the steps of:
a) obtaining a red clover (Trifolium pratense) extract comprising a plurality of aglycones;
b) determining parameters describing the rate of metabolism of the components in a plurality of mammalian tissue systems;
c) determining parameters describing distribution of the components in a plurality of mammalian tissue systems;
d) inputting the parameters into in silico models that will generate outputs to predict the pharmacokinetics and pharmacodynamics properties of the components in vivo;
e) using an optimization routine to produce a product comprising the components useful for treatment or prevention of osteoporosis.

2. The method of claim 1, further comprising the steps of determining parameters for active metabolites of the components according to steps (b) through (d), wherein results of the determinations will predict pharmacokinetics and pharmacodynamics properties of the components and their metabolites in vivo.

3. The method of claim 1, wherein the mammalian tissue systems are selected from the group consisting of gastrointestinal tract, liver, kidney, blood, mammary gland, uterus, prostate, brain, and bone.

4. The method of claim 1, wherein determining distribution of the components comprises determining enterohepatic circulation.

5. The method of claim 1, wherein the pharmacokinetics and pharmacodynamics properties comprise concentration-time profiles and response-time profiles for the components and their metabolites.

6. The method of claim 1, wherein the mathematical models are capable of solving multiple unknowns which are linearly independent or interacting with each other.

7. The method of claim 1, wherein the mathematical models comprise
$$r \approx \overline{r} + {\sum }_{i}^{ } {w}_{i} ( {d}_{i} - {\overline{d}}_{i} ) + {\sum }_{i}^{ } {{w}_{i}^{′} ( {d}_{i} - {\overline{d}}_{i} )}^{2} + {\sum }_{i , j}^{ } {w}_{i , j} ( {d}_{i} - {\overline{d}}_{i} ) ( {d}_{j} - {\overline{d}}_{j} ) ,$$
wherein r is linearized response, r̅ is the average linearized response; wᵢ is weight of the i component (relates to potency), dᵢ is the dose of component i and d̅ᵢ and d̅ⱼ are average dose of the i^{th} and j^{th} component, w_{i,j} is the weight of the interacting pair.

8. The method of claim 1, wherein the mathematical models comprise
$$A = {α}_{0} + {\sum }_{i = 1}^{n} {α}_{i} {x}_{i} + {\sum }_{i = 1}^{n} {\sum }_{j = 1}^{n} {β}_{i , j} {x}_{i} {x}_{j} ,$$
wherein α₀ and αᵢ are baseline activity and activity coefficient of component i respectively, xᵢ and xⱼ are components i and j respectively, β_{i,j} is the activity coefficient of the interacting pair, xᵢ and xⱼ, wherein said equation is able to predict an optimized composition of the extract to achieve maximum possible potency.

9. The method of claim 1, wherein the mathematical models are selected from the group consisting of least absolute shrinkage and selection operator (LASSO), wavelet-based deconvolution, compressed sensing, and gradient projection algorithm.

10. The method of claim 1, wherein the rate of metabolism comprises rate of degradation and rate of absorption.

11. The method of claim 3, wherein determining the rate of metabolism in gastrointestinal tract comprises assays using artificial gastric or intestinal juice, intestinal flora, intestinal microsomes, or permeability studies using cultured cells or intestinal tissues.

12. The method of claim 3, wherein determining the rate of metabolism in liver comprises assays using freshly harvested hepatocytes, cryopreserved hepatocytes, hepatic microsomes, hepatic cytosol or S-9 fractions.

13. The method of claim 3, wherein determining the distribution in blood comprises determining binding to plasma protein, binding to blood protein, pKa, log P, log D, and volume of distribution of a component.

14. A composition identified by the method of claim 1.

15. The composition of claim 14, comprising biochanin A, daidzein, equol and genistein.

16. The composition of claim 14, wherein biochanin A comprises between 0 to 60% of the total composition.

17. The composition of claim 14, wherein daidzein comprises between 0 to 80% of the total composition.

18. The composition of claim 14, wherein genistein comprises between 0 to 80% of the total composition.

19. The composition of claim 14, wherein equol comprises between 0 to 80% of the total composition.

20. The composition of claim 14, wherein the composition is formulated in a dosage comprising from 5 to 200 mg total phytoestrogen.

21. The composition of claim 14, wherein the composition is formulated in an immediate release dosage form.
